(19) United States
(12) Patent Application Publication
(10) Pub. No.: US 2023/0399324 A1
(43) Pub. Date: Dec. 14, 2023

(54) AGENTS FOR TREATING DISORDERS INVOLVING RYANODINE RECEPTORS

(71) Applicant: ARMGO Pharma, Inc., Ardsley, NY (US)

(72) Inventors: Sandro BELVEDERE, New York, NY (US); Jiaming YAN, New York, NY (US)

(21) Appl. No.: 18/140,969

(22) Filed: Apr. 28, 2023

Related U.S. Application Data
(63) Continuation of application No. PCT/US21/59572, filed on Nov. 16, 2021.
(60) Provisional application No. 63/114,724, filed on Nov. 17, 2020.

(51) Int. Cl. C07D 417/06; C07D 285/36; A61P 25/28

(52) U.S. Cl. CPC C07D 417/06; C07D 285/36; A61P 25/28

(57) ABSTRACT

The present disclosure relates to 1,4-benzothiazepine derivatives and use thereof to treat conditions associated with ryanodine receptors that regulate calcium channel functioning in cells.

### CROSS-REFERENCE

This application is a continuation of International Application No. PCT/US21/59572, filed Nov. 16, 2021, which claims the benefit of U.S. Provisional Application No. 63/114,724, filed Nov. 17, 2020, each of which is incorporated herein by reference in its entirety.

### BACKGROUND

The sarcoplasmic reticuluPm (SR) is a structure in cells that functions, among other things, as a specialized intracellular calcium (Ca²⁺) store. Ryanodine receptors (RyRs) are channels in the SR that open and close to regulate the release of Ca²⁺ from the SR into the intracellular cytoplasm of the cell. Release of Ca²⁺ into the cytoplasm from the SR increases cytoplasmic Ca²⁺ concentration. Open probability of RyRs refers to the likelihood that a RyR is open at any given moment, and therefore capable of releasing Ca²⁺ into the cytoplasm from the SR. Three RyR isoforms are known. RyR1 is the predominant isoform expressed in mammalian skeletal muscle, RyR2 is predominantly found in cardiac muscle, whereas RyR3 expression is low in skeletal muscle.

Ca²⁺ release from the SR is modulated by several RyR binding proteins. Calstabin1 (FKBP12) and Calstabin2 (FKBP12.6) stabilize the closed state of the RyR1 and RyR2, respectively. Mutations in RYR1 or RYR2 are characterized by reduced binding of Calstabin1 or Calstabin2, respectively, and inappropriate channel opening not related to contraction signals. This channel opening is further exacerbated by post-translational modifications such as PKA-phosphorylation, oxidation, or nitrosylation of the RyR channel. The resulting dissociation of Calstabin can lead to leaky channels, which exhibit a pathologic increase in the open probability under resting conditions. The SR Ca²⁺ leak leads to a reduction in SR Ca²⁺ content, with less Ca²⁺ available for release and consequently weaker muscle contractions.

### SUMMARY OF THE INVENTION

In some embodiments, described herein is a compound of the formula (I):

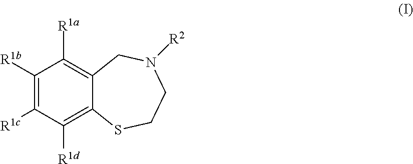

wherein
each R^{1a}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR⁷, each of which is independently substituted or unsubstituted, or hydrogen or halogen;
R² is alkyl, haloalkyl, haloalkoxy, alkenyl, alkynyl, cycloalkyl, aryl, benzyl, heteroaryl, heterocyclyl, —C(O)NR³R⁴, —C(O)C(O)NR³R⁴, —C(O)R⁸, —C(O)OR⁸, or —C(O)C(O)OR⁸, each of which is independently substituted or unsubstituted;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted; and
each R⁵, R⁶, R⁷, and R⁸ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
   or a pharmaceutically-acceptable salt thereof,
   provided that
      (a) compounds wherein (i) R^{1a}, R^{1b}, R^{1c}, and R^{1d} are each hydrogen; (ii) R^{1b} is OH or methoxy; or (iii) R² is —C(O)OtBu or —C(O)OCH₂Ph, are excluded;
      (b) when R^{1d} is methyl, then R² is not 4-methoxybenzyl; and
      (c) when R^{1a} is methyl, Cl, CN, or F, or when R^{1b} is Br, then R² is not methyl, —C(—O)H, —C(═O)Me, —C(═O)Et, or —C(═O)Ph.

In some embodiments, described herein is a pharmaceutical composition in unit dosage form comprising a pharmaceutically-acceptable excipient and a compound of the disclosure.

In some embodiments, described herein is a method of a condition, for example a condition associated with a ryanodine receptor, comprising administering to a subject in need thereof a therapeutically-effective amount of a compound of the disclosure.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DESCRIPTION OF THE DRAWINGS

FIG. 1. Compound 2 improves Calstabin2/RyR2 binding in brain microsome lysates from Huntington Disease (HD) patients.

FIG. 2. Rycals (compound 2 and reference compound S107) increase Calstabin2 binding to HD microsomes in a concentration dependent manner. ♦ Compound 2; ▪ S107.

FIG. 3. Compound 2 decreases calcium leak from HD microsomes. ♦ HD; ▪ Control; ▴ HD/Compound 2. FIG. 3a: Fluo-4 signal (% of initial signal) over time. FIG. 3b: Ca²⁺ leak (% increase in signal).

### DETAILED DESCRIPTION OF THE INVENTION

The present disclosure provides 1,4-benzothiazepine derivatives, and pharmaceutically-acceptable salts thereof. In some embodiments, the compounds are ryanodine receptor (RyR) calcium channel stabilizers, referred to as Rycals. The present disclosure further provides methods of using these compounds for treating a condition, for example a condition associated with a ryanodine receptor.

In some embodiments, compounds of the present disclosure are brain penetrant, and are suitable for treatment of central nervous system (CNS)-related disorders and conditions. In some embodiments, compounds of the disclosure are brain penetrant, metabolically stable, and pharmacologically active at treating a CNS condition associated with ryanodine receptors.

### Compounds of the Disclosure

In some embodiments, the present disclosure provides a compound of the formula (I):

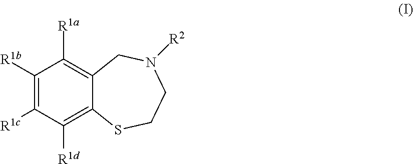

wherein
each R^{1a}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR⁷, each of which is independently substituted or unsubstituted, or hydrogen or halogen;
R² is alkyl, haloalkyl, haloalkoxy, alkenyl, alkynyl, cycloalkyl, aryl, benzyl, heteroaryl, heterocyclyl, —C(O)NR³R⁴, —C(O)C(O)NR³R⁴, —C(O)R⁸, —C(O)OR⁸, or —C(O)C(O)OR⁸, each of which is independently substituted or unsubstituted;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted; and
each R⁵, R⁶, R⁷, and R⁸ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
   or a pharmaceutically-acceptable salt thereof.

In some embodiments, compounds of formula (I) wherein R^{1a}, R^{1b}, R^{1c}, and R^{1d} are each hydrogen, are excluded. In some embodiments, compounds of formula (I) wherein R^{1b} is OH or methoxy, are excluded. In some embodiments, compounds of formula (I) wherein R² is —C(O)OtBu or —C(O)OCH₂Ph, are excluded. In some embodiments, when R^{1d} is methyl, then R² is not 4-methoxybenzyl. In some embodiments, when R^{1a} is methyl, Cl, CN, or F, or when R^{1b} is Br, then R² is not methyl, —C(═O)H, —C(═O)Me, —C(═O)Et, or —C(═O)Ph.

In some embodiments, R^{1a} is an electron withdrawing group. In some embodiments, R^{1b} is an electron withdrawing group. In some embodiments, R^{1c} is an electron withdrawing group. In some embodiments, R^{1d} is an electron withdrawing group.

In some embodiments, at least one of R^{1a}, R^{1b}, R^{1c}, and R^{1d} is haloalkyl. In some embodiments, at least one of R^{1a}, R^{1b}, R^{1c}, and R^{1d} is trifluoromethyl. In some embodiments, at least one of R^{1a}, R^{1b}, R^{1c}, and R^{1d} is halogen. In some embodiments, at least one of R^{1a}, R^{1b}, R^{1c}, and R^{1d} is fluoro. In some embodiments, at least one of R^{1a}, R^{1b}, R^{1c}, and R^{1d} is chloro. In some embodiments, at least one of R^{1a}, R^{1b}, R^{1c}, and R^{1d} is bromo. In some embodiments, at least one of R^{1a}, R^{1b}, R^{1c}, and R^{1d} is iodo. In some embodiments, at least one of R^{1a}, R^{1b}, R^{1c}, and R^{1d} is haloalkoxy. In some embodiments, at least one of R^{1a}, R^{1b}, R^{1c}, and R^{1d} is trifluoromethoxy.

In some embodiments, R^{1a} is trifluoromethyl. In some embodiments, R^{1b} is trifluoromethyl. In some embodiments, R^{1c} is trifluoromethyl. In some embodiments, R^{1d} is trifluoromethyl. In some embodiments, R^{1a} is trifluoromethoxy. In some embodiments, R^{1b} is trifluoromethoxy. In some embodiments, R^{1c} is trifluoromethoxy. In some embodiments, R^{1d} is trifluoromethoxy.

In some embodiments, R^{1a} is fluoro. In some embodiments, R^{1b} is fluoro. In some embodiments, R^{1c} is fluoro. In some embodiments, R^{1d} is fluoro. In some embodiments, R^{1a} is chloro. In some embodiments, R^{1b} is chloro. In some embodiments, R^{1c} is chloro. In some embodiments, R^{1d} is chloro.

In some embodiments, R^{1a} is bromo. In some embodiments, R^{1b} is bromo. In some embodiments, R^{1c} is bromo. In some embodiments, R^{1d} is bromo. In some embodiments, R^{1a} is iodo. In some embodiments, R^{1b} is iodo. In some embodiments, R^{1c} is iodo. In some embodiments, R^{1d} is iodo.

In some embodiments, R² is —C(O)NR³R⁴, and the compound is of formula (I)

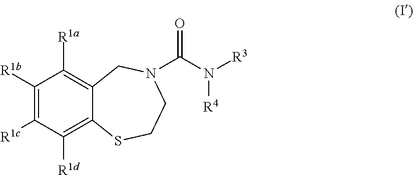

wherein
each R^{1a}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR⁷, each of which is independently substituted or unsubstituted, or hydrogen or halogen;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted; and
each R⁵, R⁶, and R⁷ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
   or a pharmaceutically-acceptable salt thereof.

In some embodiments, compounds of formula (I′) wherein (i) R^{1a}, R^{1b}, R^{1c}, and R^{1d} are each hydrogen, are excluded. In some embodiments, compounds of formula (I′) wherein R^{1b} is methoxy, are excluded.

In some embodiments, R³ and R⁴ together with the nitrogen atom to which R³ and R⁴ are attached form a heterocyclic ring, which is unsubstituted. In some embodiments, R³ and R⁴ together with the nitrogen atom to which R³ and R⁴ are attached form a heterocyclic ring, which is substituted. In some embodiments, R³ and R⁴ together with the nitrogen atom to which R³ and R⁴ are attached form a piperazinyl ring, which is unsubstituted. In some embodiments, R³ and R⁴ together with the nitrogen atom to which R³ and R⁴ are attached form a piperazinyl ring, which is substituted.

In some embodiments, the present disclosure provides a compound of formula (II)

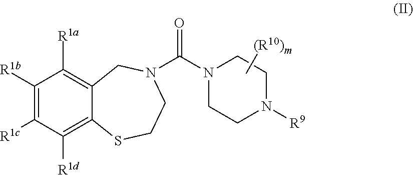

wherein
each R^{1a}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR, —SO₃H, —SO₂R⁶, —OSO₂R⁶, —S(O)R⁶, or —SR⁷, each of which is independently substituted or unsubstituted, or hydrogen or halogen;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted;
each R⁵, R⁶, and R⁷ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
R⁹ is alkyl, alkenyl, alkynyl, cycloalkyl, aryl, benzyl, heterocyclyl, heteroaryl, —C(O)NR³R⁴, —C(O)R⁸, or —C(O)OR⁸, each of which is independently substituted or unsubstituted, or hydrogen;
each R¹⁰ is independently alkyl, alkenyl, alkynyl, cycloalkyl, aryl, benzyl, heterocyclyl, heteroaryl, —NR³R⁴, —OR⁵, or —SR⁷, each of which is unsubstituted or substituted; and
m is 0, 1, 2, 3, 4, 5, 6, 7, or 8;
or a pharmaceutically-acceptable salt thereof.

In some embodiments, compounds of formula (II) wherein R^{1a}, R^{1b}, R^{1c}, and R^{1d} are each hydrogen, are excluded. In some embodiments, compounds of formula (II) wherein R^{1b} is methoxy, are excluded.

In some embodiments, the present disclosure provides a compound of formula (III)

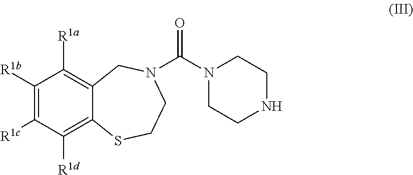

wherein
each R^{1a}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR⁷, each of which is independently substituted or unsubstituted, or hydrogen or halogen;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted;
each R⁵, R⁶, and R⁷ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
or a pharmaceutically-acceptable salt thereof.

In some embodiments, compounds of formula (III) wherein (i) R^{1a}, R^{1b}, R^{1c}, and R^{1d} are each hydrogen, are excluded. In some embodiments, compounds of formula (III) wherein R^{1b} methoxy, are excluded.

In some embodiments, the present disclosure provides a compound of the formula (IV):

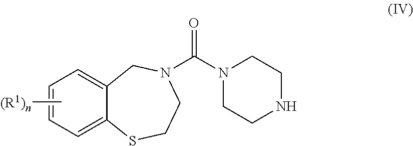

wherein
each R¹ is independently halogen, haloalkyl, haloalkyloxy; and
n is 1, 2, 3, or 4;
or a pharmaceutically-acceptable salt thereof.

In some embodiments, R¹ is halogen. In some embodiments, R¹ is fluoro. In some embodiments, R¹ is chloro. In some embodiments, R¹ is bromo. In some embodiments, R¹ is iodo. In some embodiments, R¹ is haloalkyl. In some embodiments, R¹ is halomethyl. In some embodiments, R¹ is CF₃. In some embodiments, R¹ is haloalkyloxy. In some embodiments, R¹ is halomethoxy. In some embodiments, R¹ is triflouromethoxy.

In some embodiments, n is 1. In another embodiment, n is 2. In another embodiment, n is 3. In another embodiment, n is 4.

In some embodiments, n is 1 and R¹ is at position 6 of the benzothiazepine ring, and the compound is of the formula:

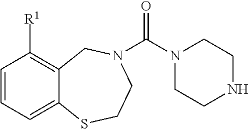

or a pharmaceutically-acceptable salt thereof.

In some embodiments, n is 1 and R¹ is at position 7 of the benzothiazepine ring, and the compound is of the formula:

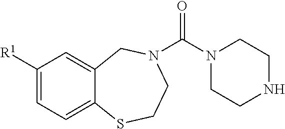

or a pharmaceutically-acceptable salt thereof.

In some embodiments, n is 1, and R¹ is at position 8 of the benzothiazepine ring, and the compound is of the formula:

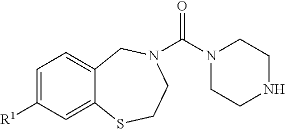

or a pharmaceutically-acceptable salt thereof.

In some embodiments, n is 1, R¹ is at position 9 of the benzothiazepine ring, and the compound is of the formula:

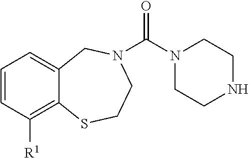

or a pharmaceutically-acceptable salt thereof.

In some embodiments, n is 2, R¹ is at positions 7 and 8 of the benzothiazepine ring, and the compound is of the formula:

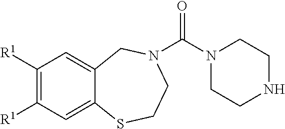

or a pharmaceutically-acceptable salt thereof.

In some embodiments, the present disclosure provides a compound that is piperazin-1-yl(8-(trifluoromethyl)-2,3-dihydrobenzo[f][1,4]thiazepin-4(5H)-yl)methanone (compound (1)), or a pharmaceutically-acceptable salt thereof. In some embodiments, the pharmaceutically-acceptable salt is a hydrochloride salt.

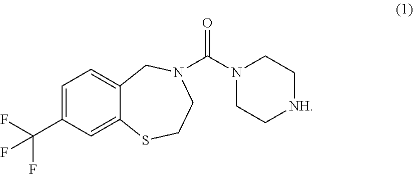 [SMILES: O=C(N1CCNCC1)N1CCSc2cc(C(F)(F)F)ccc2C1]

In some embodiments, the present disclosure provides a compound that is piperazin-1-yl(7-(trifluoromethyl)-2,3-dihydrobenzo[f][1,4]thiazepin-4(5H)-yl) methanone (compound (2)), or a pharmaceutically-acceptable salt thereof. In some embodiments, the pharmaceutically-acceptable salt is a hydrochloride salt.

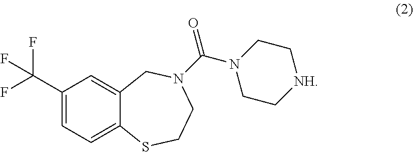 [SMILES: O=C(N1CCNCC1)N1CCSc2ccc(C(F)(F)F)cc2C1]

In some embodiments, the present disclosure provides a compound that is piperazin-1-yl(9-(trifluoromethyl)-2,3-dihydrobenzo[f][1,4]thiazepin-4(5H)-yl)methanone (compound (3)), or a pharmaceutically-acceptable salt thereof. In some embodiments, the pharmaceutically-acceptable salt is a hydrochloride salt.

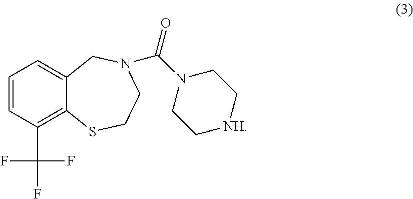 [SMILES: O=C(N1CCNCC1)N1CCSc2c(cccc2C(F)(F)F)C1]

In some embodiments, the present disclosure provides a compound that is piperazin-1-yl(7-(trifluoromethoxy)-2,3-dihydrobenzo[f][1,4]thiazepin-4(5H)-yl)methanone (compound (4)), or a pharmaceutically-acceptable salt thereof. In some embodiments, the pharmaceutically-acceptable salt is a hydrochloride salt.

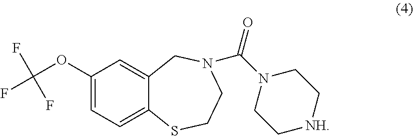 [SMILES: O=C(N1CCNCC1)N1CCSc2ccc(OC(F)(F)F)cc2C1]

In some embodiments, the present disclosure provides a compound that is piperazin-1-yl(6-(trifluoromethyl)-2,3-dihydrobenzo[f][1,4]thiazepin-4(5H)-yl)methanone (compound (5)), or a pharmaceutically-acceptable salt thereof. In some embodiments, the pharmaceutically-acceptable salt is a hydrochloride salt.

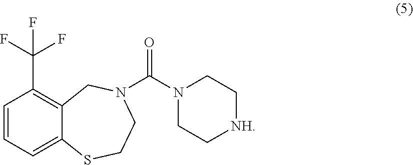 [SMILES: O=C(N1CCNCC1)N1CCSc2cccc(C(F)(F)F)c2C1]

In some embodiments, the present disclosure provides a compound that is (7,8-difluoro-2,3-dihydrobenzo[f][1,4]thiazepin-4(5H)-yl)(piperazin-1-yl)methanone (compound (6)), or a pharmaceutically-acceptable salt thereof. In some embodiments, the pharmaceutically-acceptable salt is a hydrochloride salt.

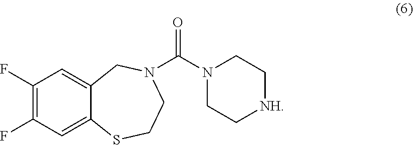 [SMILES: O=C(N1CCNCC1)N1CCSc2cc(F)c(F)cc2C1]

In some embodiments, the present disclosure provides a compound that is (6-chloro-2,3-dihydrobenzo[f][1,4]thiazepin-4(5H)-yl)(piperazin-1-yl)methanone (compound (7)), or a pharmaceutically-acceptable salt thereof. In some embodiments, the pharmaceutically-acceptable salt is a hydrochloride salt.

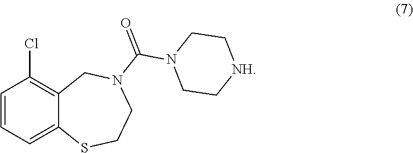 [SMILES: O=C(N1CCNCC1)N1CCSc2cccc(Cl)c2C1]

In some embodiments, the present disclosure provides a compound that is piperazin-1-yl(6-(trifluoromethoxy)-2,3-dihydrobenzo[f][1,4]thiazepin-4(5H)-yl)methanone (compound (8)), or a pharmaceutically-acceptable salt thereof. In some embodiments, the pharmaceutically-acceptable salt is a hydrochloride salt.

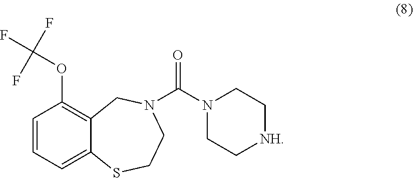 [SMILES: O=C(N1CCNCC1)N1CCSc2cccc(OC(F)(F)F)c2C1]

In some embodiments, the present disclosure provides a compound that is (6-bromo-2,3-dihydrobenzo[f][1,4]thiazepin-4(5H)-yl)(piperazin-1-yl)methanone (compound (9)), or a pharmaceutically-acceptable salt thereof. In some embodiments, the pharmaceutically-acceptable salt is a hydrochloride salt.

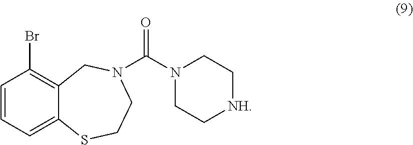 [SMILES: O=C(N1CCNCC1)N1CCSc2cccc(Br)c2C1]

In some embodiments, the present disclosure provides a compound that is (6-iodo-2,3-dihydrobenzo[f][1,4]thiazepin-4(5H)-yl)(piperazin-1-yl)methanone (compound (10)), or a pharmaceutically-acceptable salt thereof. In some embodiments, the pharmaceutically-acceptable salt is a hydrochloride salt.

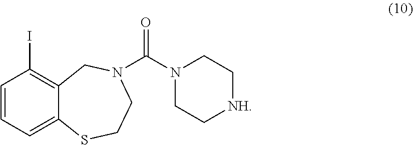 [SMILES: O=C(N1CCNCC1)N1CCSc2cccc(I)c2C1]

Several moieties described herein can be substituted or unsubstituted. Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, ureido groups, epoxy groups, and ester groups. Other non-limiting examples of optional substituents include halogen, haloalkyl, hydroxy, alkoxy, haloalkoxy, cycloalkyl, aryl, heterocyclyl, heteroaryl, amido, alkylamido, dialkylamido, nitro, amino, cyano, azido, oxo, alkylamino, dialkylamino, carboxyl, thio, thioalkyl and thioaryl.

Non-limiting examples of alkyl groups include straight, branched, and cyclic alkyl groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, isopropyl, n-butyl, isobutyl, sec-butyl, tert-butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl. Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl.

Non-limiting examples of cyclic alkyl groups include cyclopropyl, cyclobutyl, cyclopentyl, cyclohexyl, cycloheptyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl or alkynylene groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo group can be, for example, a chloro, bromo, fluoro, or iodo. A haloalkyl group can be any alkyl group substituted with any number of halogen atoms, for example, fluorine, chlorine, bromine, and iodine atoms. A haloalkenyl group can be any alkenyl group substituted with any number of halogen atoms. A haloalkynyl group can be any alkynyl group substituted with any number of halogen atoms.

Non-limiting examples of a haloalkyl group are trifluoromethyl, trichloromethyl, tribromomethyl, triiodomethyl, difluoromethyl, chlorodifluoromethyl, pentafluoroethyl, 1,1-difluoroethyl bromomethyl, chloromethyl, fluoromethyl, and iodomethyl.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy. Alkoxy groups can be, for example, substituted or unsubstituted. Alkoxy group can be substituted for example, with oxo, halogen, amino, nitrile, nitro, hydroxyl, haloalkyl, alkoxy, aryl, cycloalkyl, heterocycloalkyl, and heteroaryl.

A haloalkoxy group is an alkoxy group that is substituted by one or more halogen atoms, i.e., F, Cl, Br, or I. Non-limiting examples of haloalkoxy groups include trifluoromethoxy, trichloromethoxy, tribromomethoxy, triiodomethoxy, trifluoroethoxy, trichloroethoxy, tribromoethoxy, triiodoethoxy, trifluoropropoxy, trichlorompropoxy, tribromopropoxy, triiodopropoxy, trifluoroisopropoxy, trichloromisopropoxy, tribromoisopropoxy, triiodoisopropoxy, trifluoroisobutoxy, trichloromisobutoxy, tribromoixobutoxy, and triiodoisobutoxy. A haloalkoxy group can be substituted, for example, with amino, nitrile, nitro, hydroxyl, haloalkyl, alkoxy, aryl, cycloalkyl, heterocycloalkyl, or heteroaryl. For example, a halogen or hydrogen group of a haloalkoxy group can be optionally replaced by amino, nitrile, nitro, hydroxyl, haloalkyl, alkoxy, aryl, cycloalkyl, heterocycloalkyl, or heteroaryl.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl. Non-limiting examples of substituted aryl groups include 3,4-dimethylphenyl, 4-tert-butylphenyl, 4-cyclopropylphenyl, 4-diethylaminophenyl, 4-(trifluoromethyl)phenyl, 4-(difluoromethoxy)-phenyl, 4-(trifluoromethoxy)phenyl, 3-chlorophenyl, 4-chlorophenyl, 3,4-dichlorophenyl, 2-fluorophenyl, 2-chlorophenyl, 2-iodophenyl, 3-iodophenyl, 4-iodophenyl, 2-methylphenyl, 3-fluorophenyl, 3-methylphenyl, 3-methoxyphenyl, 4-fluorophenyl, 4-methylphenyl, 4-methoxyphenyl, 2,3-difluorophenyl, 3,4-difluorophenyl, 3,5-difluorophenyl, 2,3-dichlorophenyl, 3,4-dichlorophenyl, 3,5-dichlorophenyl, 2-hydroxyphenyl, 3-hydroxyphenyl, 4-hydroxyphenyl, 2-methoxyphenyl, 3-methoxyphenyl, 4-methoxyphenyl, 2,3-dimethoxyphenyl, 3,4-dimethoxyphenyl, 3,5-dimethoxyphenyl, 2,4-difluorophenyl, 2,5-difluorophenyl, 2,6-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 2,4-dichlorophenyl, 2,5-dichlorophenyl, 2,6-dichlorophenyl, 3,4-dichlorophenyl, 2,3,4-trichlorophenyl, 2,3,5-trichlorophenyl, 2,3,6-trichlorophenyl, 2,4,5-trichlorophenyl, 3,4,5-trichlorophenyl, 2,4,6-trichlorophenyl, 2,3-dimethylphenyl, 2,4-dimethylphenyl, 2,5-dimethylphenyl, 2,6-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 2,3,4-triethylphenyl, 2,3,5-triethylphenyl, 2,3,6-triethylphenyl, 2,4,5-triethylphenyl, 2,4,6-triethylphenyl, 2-isopropylphenyl, 3-isopropylphenyl, and 4-isopropylphenyl.

Non-limiting examples of substituted aryl groups include 2-aminophenyl, 2-(N-methylamino)phenyl, 2-(N,N-dimethylamino)phenyl, 2-(N-ethylamino)phenyl, 2-(N,N-diethylamino)phenyl, 3-aminophenyl, 3-(N-methylamino)phenyl, 3-(N,N-dimethylamino)phenyl, 3-(N-ethylamino)phenyl, 3-(N,N-diethylamino)phenyl, 4-aminophenyl, 4-(N-methylamino)phenyl, 4-(N,N-dimethylamino)phenyl, 4-(N-ethylamino)phenyl, and 4-(N,N-diethylamino)phenyl.

An aryloxy group can be, for example, an oxygen atom substituted with any aryl group. An ether or an ether group comprises an aryloxy group. The aryloxy group can be substituted or unsubstituted. An aryloxy group can be substituted, for example, with amino, nitrile, nitro, hydroxyl, haloalkyl, alkoxy, aryl, cycloalkyl, heterocycloalkyl, or heteroaryl. For example, a halogen or hydrogen group of a haloalkoxy group can be optionally replaced by amino, nitrile, nitro, hydroxyl, haloalkyl, alkoxy, aryl, cycloalkyl, heterocycloalkyl, or heteroaryl.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic.

Non-limiting examples of heterocycles include piperazine, pyrrole, pyrrolidine, pyridine, piperidine, succinamide, maleimide, morpholine, imidazole, thiophene, furan, tetrahydrofuran, pyran, and tetrahydropyran.

Non-limiting examples of heterocycles (heterocyclyl) include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

An amine is a group NH₂. An alkylamine is an amine substituted by one or more alkyl groups.

An arylamine is an amine that is substituted by one or more alkyl groups. An heterocyclylamine is an amine substituted by one or more heterocyclic groups. A heteroarylamine is an amine substituted by one or more heteroaryl groups.

In some embodiments, a compound exists in a population of tautomeric forms. All such tautomeric forms are contemplated herein as part of the present disclosure.

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

Subsequent to preparation, compounds of the present disclosure can be isolated and purified to obtain a composition containing an amount by mass of at least about 90%, at least about 91%, at least about 92%, at least about 93%, at least about 94%, at least about 95%, at least about 96%, at least about 97%, at least about 98%, at least about 99% or about 100% of the compound or a salt thereof.

### Pharmaceutically Acceptable Salts

Any compound herein can be provided as a pharmaceutically-acceptable salt. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt.

Metal salts can arise from the addition of an inorganic base to a compound of the present disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, imidazole, or pyrazine.

In some embodiments, an ammonium salt is a triethyl amine salt, a trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, or a maleate salt.

In some embodiments, one or more of the compounds of the disclosure is in the form of a salt protonated on a nitrogen atom, including salts formed with organic and inorganic anions and cations discussed herein. Non-limiting examples of such acids include hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicyclic, methanesulfonic, benzenesulfonic, sorbic, picric, benzoic, and cinnamic acid.

### Therapeutic Uses

In some embodiments, the present disclosure provides a compound capable of treating conditions, disorders, and diseases associated with Ryanodine Receptors (RyRs).

In some embodiments, the present disclosure provides compounds that are RyR modulators, for example, a Rycal compound. Rycal compounds are small molecules that can, for example, bind to leaky RyR subunits, restore Calstabin binding, and repair the channel leak. In some embodiments, Rycals bind to leaky RyR channels, restore Calstabin binding, and fix the channel leak without blocking the RyR channel. In some embodiments, Rycal compounds are capable of fixing a leak in RyR channels, for example, RyR1, RyR2, and/or RyR3 channels. In some embodiments, the compositions of the disclosure enhance association and/or inhibit dissociation of RyR and Calstabin (e.g., RyR1 and Calstabin1; RyR2 and Calstabin2; and RyR3 and Calstabin1).

Non-limiting examples of conditions, disorders, and diseases associated with RyRs include disorders and diseases that can be treated and/or prevented by modulating RyRs and include, for example, a cardiac disorder or disease, a musculoskeletal disorder or disease, cancer associated muscle weakness, malignant hyperthermia, and diabetes. A compound herein can also lessen the likelihood of the occurrence of such a condition.

In some embodiments, the present disclosure provides a method of treating or reducing a likelihood of occurrence of a condition by administering to a subject in need thereof a therapeutically-effective amount of a compound disclosed herein, e.g., a compound of formula (I), formula (I′), formula (II), formula (III), or formula (IV) as described herein, or a pharmaceutically-acceptable salt thereof. In some embodiments, the compound is administered in a pharmaceutical composition. In some embodiments, the compound is in a unit dosage form. In some embodiments, the unit dosage form is a solid dosage form. In some embodiments, the pharmaceutical composition is in a unit dosage form suitable for oral administration.

In some embodiments, the present disclosure provides a compound, e.g., a compound of formula (I), formula (I′), formula (II), formula (III), or formula (IV) as described herein, or a pharmaceutically-acceptable salt thereof, for use in a method of treating or reducing a likelihood of occurrence of a condition.

In some embodiments, the present disclosure a compound, e.g., a compound of formula (I), formula (I′), formula (II), formula (III), or formula (IV) as described herein, or a pharmaceutically-acceptable salt thereof, for use in the manufacture of a medicament.

In some embodiments, the condition, disorder, or disease is associated with an abnormal function of RyR1. In some embodiments, the condition, disorder, or disease is associated with an abnormal function of RyR2. In some embodiments, the condition, disorder or disease is associated with an abnormal function of RyR3.

In some embodiments, the present disclosure provides a method of modulating the binding of RyRs and Calstabins in a subject, including administering to the subject an amount of a compound, e.g., a compound of formula (I), formula (I′), formula (II), formula (III), or formula (IV) as described herein, or a salt thereof, effective to modulate the amount of RyR-bound Calstabin. In some embodiments, the compound is used at a dose sufficient to restore or enhance binding of Calstabin2 to RyR2. In other embodiments, the compound is used at a dose sufficient to restore or enhance binding of Calstabin2 to RyR2. In some embodiments, the compound is used at a dose sufficient to restore or enhance binding of Calstabin1 to RyR1. In other embodiments, the compound is used at a dose sufficient to restore or enhance binding of Calstabin1 to RyR1.

Methods of the disclosure can be practiced on an in vitro system (e.g., cultured cells or tissues) or in vivo (e.g., in a non-human animal or a human).

### Ryanodine Receptors: Excitation-Contraction Coupling (ECC) Process

The sarcoplasmic reticulum (SR) is a structure in cells that functions, among other things, as a specialized intracellular calcium (Ca²⁺) store. Ryanodine receptors (RyRs) are channels in the SR, which open and close to regulate the release of Ca²⁺ from the SR into the intracellular cytoplasm of the cell. Release of Ca²⁺ into the cytoplasm from the SR increases cytoplasmic Ca²⁺ concentration. Open probability of RyRs refers to the likelihood that a RyR is open at any given moment, and therefore capable of releasing Ca²⁺ into the cytoplasm from the SR.

The RyR is the major Ca²⁺ release channel on the SR responsible for excitation-contraction coupling (ECC) in striated muscle. Among the three known RyR isoforms (RyR1, RyR2 and RyR3), RyR1 is widely expressed and is the predominant isoform expressed in mammalian skeletal muscle. RyR2 is also widely expressed and is the predominant form found in cardiac muscle. RyR3 expression is low in adult skeletal muscle. RyR subtypes exhibit a high degree of structural and functional homology. The subtypes form a large sarcoplasmic membrane complex, consisting of four monomers that constitute a Ca²⁺ release channel associated with proteins, such as kinases, phosphatases, phosphodiesterases, and other regulatory subunits.

Ca²⁺ release from the SR is modulated by several RyR binding proteins. Calmodulin, a key mediator of Ca²⁺ signaling, exerts both positive and negative effects on RyR open probability. Calstabin1 (FKBP12) and calstabin2 (FKBP12.6) stabilize the closed state of RyR1 and RyR2, respectively. Calstabin1 associates predominantly with skeletal muscle RyR1, while cardiac muscle RyR2 has the highest affinity for Calstabin2.

Mutations in RYR1 or RYR2 can cause decreased binding of Calstabin1 and Calstabin2, respectively. Stress-induced post-translational modifications of RyRs including PKA phosphorylation, oxidation, and nitrosylation also can cause decreased binding of Calstabins to RyR channels. Genetic mutations and/or stress-induced posttranslational modifications of the channel can result in dissociation of Calstabin from RyRs and cause the channels to become leaky channels. The dissociation of Calstabin can lead to leaky channels, which exhibit a pathologic increase in the open probability under resting conditions. The SR Ca²⁺ leak leads to a reduction in SR Ca²⁺ content, with less Ca²⁺ available for release and consequently weaker muscle contractions. The intracellular calcium leak has distinct pathological consequences depending on which tissue is involved.

### Ryanodine Receptors and Disorders of the Central Nervous System

In some embodiments, pharmaceutical compositions described herein is administered to a subject in need thereof. In some embodiments, the subject in need thereof has a condition or disease. In some embodiments, the pharmaceutical composition described herein is administered to treat a subject in need thereof with a condition or disease, wherein the pharmaceutical composition herein reduces a symptom or symptoms of the condition or disease.

In some embodiments, the RyR-associated condition is a central nervous system (CNS) disorder or disease that implicates the Ryanodine Receptor 1 (RyR1). In some embodiments, the RyR-associated condition is a central nervous system (CNS) disorder or disease that implicates the Ryanodine Receptor 2 (RyR2). In some embodiments, the RyR-associated condition is a central nervous system (CNS) disorder or disease that implicates the Ryanodine Receptor 3 (RyR3). In some embodiments, the condition is a peripheral central nervous system condition, disorder or disease. In some embodiments, the condition is a neurological condition, disorder or disease. In some embodiments, the condition is a neurodegenerative disease. In some embodiments, the condition is cognitive dysfunction. In some embodiments, compounds of the disclosure are useful in improving cognitive function. In some embodiments, compounds of the disclosure are useful in treating of cognitive dysfunction. In some embodiments, compounds of the disclosure are useful in slowing progression of cognitive dysfunction. In some embodiments, compounds of the disclosure are useful in reducing likelihood of occurrence of cognitive dysfunction.

In some embodiments, the present disclosure relates to a method of treating or reducing the likelihood of occurrence of conditions, disorders, and diseases of the nervous system, by administering to a subject in need thereof an amount of a compound described herein, e.g., a compound of Formula (I), a compound of Formula (I′), a compound of formula (II), a compound of formula (I), a compound of formula (IV), or a pharmaceutically-acceptable salt thereof, or a pharmaceutical composition comprising such compound.

In some embodiments, the present disclosure relates to the use of a compound described herein, e.g., a compound of Formula (I), a compound of Formula (I′), a compound of formula (II), a compound of formula (III), a compound of formula (IV), or a pharmaceutically-acceptable salt thereof, or a pharmaceutical composition comprising such compound, for treating or reducing the likelihood of occurrence of conditions, disorders, and diseases of the nervous system.

In another embodiment, the present disclosure relates to a compound described herein, e.g., a compound of Formula (I), a compound of Formula (I′), a compound of formula (II), a compound of formula (III), a compound of formula (IV), or a pharmaceutically-acceptable salt thereof, or a pharmaceutical composition comprising such compound, for use in treating or reducing the likelihood of occurrence of conditions, disorders, and diseases of the nervous system.

In some embodiments, conditions, disorders, and diseases treatable or preventable by the compounds of the disclosure include Alzheimer's disease, post-traumatic stress disorder (PTSD), Huntington's Disease, neuropathy, seizure disorders, Amyotrophic lateral sclerosis (ALS, Lou Gehrig's disease), Spinocerebellar ataxia, and Parkinson's Disease.

In some embodiments, compounds of the present disclosure are useful for treating a movement disorder. Non-limiting examples of movement disorders include ataxia, dystonia, chorea, Huntington's disease, functional movement disorder, multiple system atrophy, Parkinson's disease, Parkinsonism, a movement disorder due to Alzheimer's disease, progressive supranuclear palsy, restless legs syndrome, tardive dyskinesia, Tourette syndrome, tremors, and Wilson's disease.

In some embodiments, the movement disorder is or is characterized by a tremor. Non-limiting examples of tremors include essential tremor, Parkinsonism tremor, dystonic tremor, cerebellar tremor, psychogenic tremor, orthostatic tremor, and physiologic tremor.

In some embodiments, the movement disorder is essential tremor. Essential tremor is a tremor predominantly present in bilateral upper extremities, or less commonly in other locations, such as the head, neck, vocal cords, or lower limbs. Essential tremor is one of the most common movement disorders and tends to worsen with age. Characteristically, essential tremor is more pronounced upon attempts to use the upper extremities, rather than at rest. Consequently, hand writing or drawing difficulties are often marked.

Other examples of neurodegenerative diseases include Parkinson-like Disease, Multiple Sclerosis, autoimmune disorders, Pick Disease, diffuse Lewy body Disease, progressive supranuclear palsy (Steel-Richardson syndrome), multisystem degeneration (Shy-Drager syndrome), motor neuron diseases, amyotrophic lateral sclerosis, degenerative ataxias, cortical basal degeneration, ALS-Parkinson-Dementia complex of Guam, subacute sclerosing panencephalitis, synucleinopathies, primary progressive aphasia, striatonigral degeneration, Machado-Joseph disease/spinocerebellar ataxia type 3 and olivopontocerebellar degenerations, Gilles De La Tourette Disease, bulbar and pseudobulbar palsy, spinal and spinobulbar muscular atrophy (Kennedy Disease), primary lateral sclerosis, familial spastic paraplegia, Werdnig-Hoffmann Disease, Kugelberg-Welander Disease, Tay-Sach Disease, Sandhoff Disease, familial spastic disease, Wohlfart-Kugelberg-Welander Disease, spastic paraparesis, progressive multifocal leukoencephalopathy, prion diseases, including Creutzfeldt-Jakob Disease, Gerstmann-Straussler-Scheinker Disease, Kuru, and fatal familial insomnia.

Neurodegenerative diseases also include ischemic and hemorrhagic stroke, spinal cord injury, brain injury, Schizophrenia, Autism, Ataxia, Amyotrophic Lateral Sclerosis, Lou Gehrig's Disease, Lyme Disease, Meningitis, Migraine, Motor Neuron Diseases, pain, brain damage, brain dysfunction, spinal cord disorders, peripheral nervous system disorders, cranial nerve disorders, autonomic nervous system disorders, sleep disorders, headaches, lower back and neck pain, neuropathic pain, dementia, delirium and dementia dizziness and vertigo, stupor and coma, head injury, stroke, tumors of the nervous system, infections of the brain or spinal cord, prion diseases, depression, and drug addiction.

Dementia refers to decline in cognitive function due to damage or disease in the brain or central nervous system beyond that which might be expected from normal aging. Dementias typically affect cognitive functions such as learning, memory, attention, language skills, and problem solving skills. Types and causes of dementia include Alzheimer's disease, vascular dementia (also known as multiinfarct dementia), Binswanger's disease, dementia with Lewy bodies (DLB), alcohol-induced persisting dementia, frontotemporal lobar degenerations (FTLD), Pick's disease, frontotemporal dementia (or frontal variant FTLD), semantic dementia (or temporal variant FTLD), progressive non-fluent aphasia, Creutzfeldt-lakob disease, Huntington's disease, Parkinson's di and AIDS dementia complex.

“Amyotrophic lateral sclerosis” or “ALS” is a progressive neurodegenerative disease that affects upper motor neurons (motor neurons in the brain) and/or lower motor neurons (motor neurons in the spinal cord) and results in motor neuron death. Non-limiting examples of ALS include classical ALS (typically affecting both lower and upper motor neurons), Primary Lateral Sclerosis (PLS, typically affecting only the upper motor neurons), Progressive Bulbar Palsy (PBP or Bulbar Onset, a version of ALS that typically begins with difficulties swallowing, chewing and speaking), Progressive Muscular Atrophy (PMA, typically affecting only the lower motor neurons), and familial ALS (a genetic version of ALS).

“Multiple sclerosis” or “MS” is a progressive neurodegenerative disease resulting in destruction of the myelin covering of nerve cells, particularly of the brain and spinal cord. Non-limiting examples of multiple sclerosis include Relapsing-remitting (RRMS) (typically characterized by partial or total recovery after attacks (also called exacerbations, relapses, or flares)), Secondary progressive (SPMS) (generally characterized by fewer relapses, with an increase in disability and symptoms), and Primary progressive (PPMS) (generally characterized by progression of symptoms and disability without remission).

“Alzheimer's disease” or “AD” is a progressive neurodegenerative disease characterized by dementia and defined by the American Psychiatric Association (in DSM IV) as the development of multiple cognitive deficits that includes memory impairment.

Parkinson's disease is a neurodegenerative disease. Many of the signs and symptoms associated with Parkinson's disease can precede typical Parkinson's disease, in some cases by many years. Involvement of the dopaminergic substantia nigra, which underlies the primary motor features of the disease, occurs at a time when the disease is well advanced at a neuropathological level. The motor features of Parkinson's disease are characterized by muscle rigidity, tremor, gait and postural abnormalities, a slowing of physical movement (bradykinesia) and, in extreme cases, a loss of physical movement (akinesia). The primary symptoms are the results of decreased stimulation of the motor cortex and other areas of the brain by the basal ganglia, normally caused by the insufficient formation and action of dopamine, which is produced in the dopaminergic neurons of the brain. The motor features of Parkinson's disease are just one component of a much more wide-spread disorder that causes an abundance of non-motor signs and symptoms, including olfactory dysfunction, REM sleep behavioral disorder (RBD), constipation, depression, and cognitive deficits. Many of these signs and symptoms can precede the motor symptoms by years to a decade or more.

Parkinson's-Like Diseases: several other conditions have the features of Parkinson's disease and are interchangeably referred to as Parkinson's-like disease, secondary Parkinsonism, Parkinson's syndrome, or atypical Parkinson's. These neurological syndromes can be characterized by tremor, hypokinesia, rigidity, and postural instability. Several etiologies can lead to similar symptoms, including some toxins, metabolic diseases, and non-PD neurological conditions. A common cause is as a side effect of medications, mainly neuroleptic antipsychotics, especially the phenothiazines (such as perphenazine and chlorpromazine), thioxanthenes (such as flupenthixol and zuclopenthixol) and butyrophenones (such as haloperidol (Haldol)), piperazines (such as ziprasidone), and rarely, antidepressants. Other causes include but are not limited to olivopontocerebellar degeneration; progressive supranuclear palsy; corticobasal degeneration; temporo-frontal dementia; drug-induced by antipsychotics, prochlorperazine, or metoclopromide; poisoning with carbon monoxide; head trauma; and Huntington's disease Parkinsonism. In some cases alpha-synucleinopathies can result in Parkinson's-like disease, secondary Parkinsonism, Parkinson's syndrome, or atypical Parkinson's. In some embodiments, the methods described herein are used to diagnose Parkinson's-like disease, secondary Parkinsonism, and Parkinson's syndrome.

### Cognitive Dysfunction

In some embodiments, compounds of the disclosure are useful in treating cognitive dysfunction. In some embodiments, the present disclosure relates to a method of treating or reducing the likelihood of occurrence cognitive dysfunction, or for improving cognitive function, by administering to a subject in need thereof an amount of a compound described herein, e.g., a compound of Formula (I), a compound of formula (I-A), a compound of formula (II), or a compound of formula (III), or a pharmaceutically-acceptable salt thereof, or a pharmaceutical composition comprising such compound.

In some embodiments, the present disclosure relates to the use of a compound described herein, e.g., a compound of Formula (I), a compound of formula (I-A), a compound of formula (II), or a compound of formula (III), or a pharmaceutically-acceptable salt thereof, or a pharmaceutical composition comprising such compound, for treating or reducing the likelihood of occurrence of cognitive dysfunction, or for improving cognitive function.

In some embodiments, the present disclosure relates to a compound described herein, e.g., a compound of Formula (I), a compound of formula (I-A), a compound of formula (II), or a compound of formula (III), or a pharmaceutically-acceptable salt thereof, or a pharmaceutical composition comprising such compound, for use in treating or reducing the likelihood of occurrence of cognitive dysfunction, or for improving cognitive function.

In some embodiments, cognitive dysfunction is associated with stress-related cognitive dysfunction or age-related cognitive dysfunction or a combination thereof. In some embodiments, cognitive dysfunction is associated with a disease. Non-limiting examples of diseases implicated with cognitive dysfunction are post-traumatic stress disorder, attention deficit hyperactivity disorder, autism spectrum disorder, generalized anxiety disorder, obsessive compulsive disorder, Schizophrenia, Bipolar disorder, Parkinson's disease, and major depression.

In some embodiments, the compounds of the present disclosure improve cognitive function, for example, short term memory, long term memory, attention, learning, and any combination thereof.

### Ryanodine Receptor 2 and Cardiac Diseases

In some embodiments, the RyR-associated condition is a cardiac disorder or disease that implicates the Ryanodine Receptor 2 (RyR2). The RyR2 channel plays a major role in intracellular calcium handling by regulating the release of Ca²⁺ from the sarcoplasmic reticulum (SR) in cardiac myocytes required for ECC in cardiac muscle. The RyR2 channel is a macromolecular complex, which includes four identical RyR2 subunits, each of which binds one Calstabin2 (FKBP12.6), and other interacting proteins such as phosphatases and kinases. Binding of Calstabin2 stabilizes the channel in the closed state during the resting phase of the heart (diastole), thereby preventing diastolic calcium leak from the SR, and functionally couples groups of RyR2 channels to allow synchronous opening during excitation-contraction coupling.

Phosphorylation of RyR2 by protein kinase A (PKA) is an important part of the fight-or-flight response. Phosphorylation increases cardiac EC coupling gain by augmenting the amount of Ca²⁺ released for a given trigger. The process strengthens muscle contraction and improves exercise capacity. This signaling pathway provides a mechanism by which activation of the sympathetic nervous system (SNS), in response to stress, results in increased cardiac output. Phosphorylation of RyR2 by PKA increases the sensitivity of the channel to calcium-dependent activation. The increased sensitivity leads to increased open probability and increased calcium release from the SR into the intracellular cytoplasm.

Heart failure (HF) is characterized by a sustained hyperadrenergic state in which serum catecholamine levels are chronically elevated. One consequence of this chronic hyperadrenergic state is persistent PKA hyperphosphorylation of RyR2, such that 3-4 out of the four Ser2808 in each homotetrameric RyR2 channel are chronically phosphorylated. Chronic PKA hyperphosphorylation of RyR2 is associated with depletion of the channel-stabilization subunit Calstabin2 from the RyR2 channel macromolecular complex. Depletion of Calstabin2 results in a diastolic SR Ca²⁺ leak from the RyR complex, and contributes to impaired contractility. Due to the activation of inward depolarizing currents, this diastolic SR Ca²⁺ leak also is associated with fatal cardiac arrhythmias. Mice engineered with RyR2 lacking the PKA phosphorylation site (RyR-S2808A) are protected from HF progression after myocardial infarction (MI). In addition, chronic PKA hyperphosphorylation of RyR2 in HF is associated with remodeling of the RyR2 macromolecular complex. The remodeling includes depletion of phosphatases PP1 and PP2a (impairing dephosphorylation of Ser2808) and the cAMP-specific type 4 phosphodiesterase (PDE4D3) from the RyR2 complex. Depletion of PDE4D3 from the RyR2 complex causes sustained elevation of local cAMP levels. Thus, diastolic SR Ca²⁺ leak contributes to HF progression and arrhythmias. Additional post-translational modifications of the RyR channel (oxidation and nitrosylation) further drive the leak.

RyR leak is associated with a variety of cardiac disorders, conditions, and diseases. In some embodiments, the cardiac disorder or disease is heart failure. In some embodiments, the cardiac disorder or disease is myocardial infarction (MI). In some embodiments, the heart failure is congestive heart failure. In some embodiments, the heart failure is chronic heart failure. In some embodiments, the heart failure is systolic heart failure. In some embodiments, the heart failure is diastolic heart failure. In some embodiments, the heart failure is acute decompensated heart failure. In some embodiments, the heart failure is heart failure with reduced or preserved ejection fraction. In some embodiments, the heart failure is acute heart failure, for example, for preservation of cardiac function post myocardial infarction or cardiomyopathy.

In some embodiments, the cardiac disorder or disease comprises cardiac ischemia/reperfusion (I/R) injury. I/R injury can occur following coronary angioplasty or following thrombolysis for the treatment of myocardial infarction (MI) or during/following cardiac bypass surgery or heart transplant.

In some embodiments, the cardiac disorder or disease is characterized by an irregular heartbeat or an arrhythmia. In some embodiments, the cardiac disorder or disease is catecholaminergic polymorphic ventricular tachycardia (CPVT). In some embodiments, the cardiac disorder or disease is, or is characterized by, an atrial arrhythmia. In some embodiments, the cardiac disorder or disease is, or is characterized by, a ventricular arrhythmia. In some embodiments, the cardiac disorder or disease is, or is characterized by, atrial fibrillation. In some embodiments, the cardiac disorder or disease is, or is characterized by, ventricular fibrillation. In some embodiments, the cardiac disorder or disease is, or is characterized by, atrial tachyarrhythmia. In some embodiments, the cardiac disorder or disease is, or is characterized by, ventricular tachyarrhythmia. In some embodiments, the cardiac disorder or disease is, or is characterized by, atrial tachycardia. In some embodiments, the cardiac disorder or disease is, or is characterized by, ventricular tachycardia. In some embodiments, the cardiac disorder or disease is, or is characterized by, sick sinus syndrome. In some embodiments, the cardiac disorder or disease is, or is characterized by, Sudden infant death syndrome (SDIS). In some embodiments, the cardiac disorder or disease is, or is characterized by, sudden unexplained death (SUD).

In some embodiments, the cardiac disorder or disease is Catecholaminergic Polymorphic Ventricular Tachycardia (CPVT). CPVT is one of the most lethal inherited arrhythmogenic disorders. CPVT occurs in the absence of structural heart disease and is characterized by adrenergically mediated ventricular arrhythmias associated with a high incidence of Sudden Cardiac Death (SCD). Patients usually present in the first or second decade of life with stress-induced syncope. CPVT is associated with mutations in two genes that code for proteins associated with the sarcoplasmic reticulum (SR) of the cardiomyocyte. The most frequently observed Form is CPVT type 1, an autosomal dominant form due to mutations in RyR2. This type encodes an intracellular SR calcium release channel. CPVT-associated RyR2 mutations result in leaky RyR2 channels due to the decreased binding of the Calstabin2 (FKBP12.6) subunit, which stabilizes the closed state of the channel. Mice heterozygous for the R2474S mutation (which occurs in humans with CPVT1) in RyR2 (RyR2-R2474S mice) can exhibit exercise-induced ventricular arrhythmias and sudden cardiac death. Treatment with Rycals that enhance the binding of Calstabin2 to the mutant RyR2-R2474S channel can inhibit the channel leak and prevent cardiac arrhythmias.

### Ryanodine Receptor 1 and Musculoskeletal Diseases

In some embodiments, the RyR-associated condition is a musculoskeletal disorder or disease that implicates the Ryanodine Receptor 1 (RyR1). The RyR1 macromolecular complex consists of a tetramer of the 560-kDa RyR1 subunit that forms a scaffold for proteins that regulate channel function including PKA and the phosphodiesterase 4D3 (PDE4D3), protein phosphatase 1 (PP1) and Calstabin1. A-kinase anchor protein (mAKAP) targets PKA and PDE4D3 to RyR1, whereas spinophilin targets PP1 to the channel. The catalytic and regulatory subunits of PKA, PP1, and PDE4D3 regulate PKA-mediated phosphorylation of RyR1 at Ser2843 (Ser2844 in the mouse). PKA-mediated phosphorylation of RyR1 at Ser2844 increases the sensitivity of the channel to cytoplasmic Ca²⁺, reduces the binding affinity of Calstabin1 for RyR1, and destabilizes the closed state of the channel.

Calstabin1 concentrations in skeletal muscle can be approximately 200 nM. PKA phosphorylation of RyR1 can reduce the binding affinity of Calstabin1 for RyR1 from approximately 100-200 nM to more than 600 nM. Thus, under physiologic conditions, reduction in the binding affinity of Calstabin1 for RyR1, resulting from PKA phosphorylation of RyR1 at Ser2843, is sufficient to reduce substantially the amount of Calstabin1 present in the RyR1 complex. Chronic PKA hyperphosphorylation of RyR1 at Ser2843) results in leaky channels (i.e., channels prone to opening at rest), which contribute to the skeletal muscle dysfunction that is associated with persistent hyperadrenergic states such as those in individuals with heart failure.

Moreover, regulation of RyR1 by posttranslational modifications other than phosphorylation, such as by nitrosylation of free sulfhydryl groups on cysteine residues (S-nitrosylation), and channel oxidation, can increase RyR1 channel activity. S-nitrosylation and oxidation of RyR1 each can reduce Calstabin1 binding to RyR1.

In some embodiments, the musculoskeletal disorder or disease is a congenital myopathy or congenital muscular dystrophy (CMD). Congenital muscular dystrophy is present at birth. CMD is classified based on genetic mutations: 1) genes encoding for structural proteins of the basal membrane or extracellular matrix of the skeletal muscle fibers; 2) genes encoding for putative or demonstrated glycosyltransferases, that in turn affect the glycosylation of dystroglycan, an external membrane protein of the basal membrane; and 3) other. Non-limiting examples of CMD include RYR1-related myopathies (RYR1-RM), Laminin-α2-deficient CMD (MDC1A), Ullrich CMG (UCMDs 1, 2 and 3), Walker-Warburg syndrome (WWS), Muscle-eye-brain disease (MEB), Fukuyama CMD (FCMD), CMD plus secondary laminin deficiency 1 (MDC1B), CMD plus secondary laminin deficiency 2 (MDC1C), CMD with mental retardation and pachygyria (MDC1D), and Rigid spine with muscular dystrophy Type 1 (RSMD1).

In some embodiments, the musculoskeletal disease is RYR1-related congenital myopathy (RYR1-RM). RYR1-RM comprise a group of rare neuromuscular diseases. Affected individuals generally present with delayed motor milestones, muscle weakness, impaired ambulation, and, in severe cases, scoliosis, ophthalmoplegia, and respiratory distress all due to skeletal muscle weakness. Causative variants in RYR1, which encodes the major calcium (Ca²⁺) release channel in skeletal muscle, exert different effects on the RyR1 channel. The variants generally disrupt the normal Ca²⁺ flow between the sarcoplasmic reticulum (SR) and muscle cell cytosol and commonly result in excessive Ca²⁺ leak into the cytosol. Persistent Ca²⁺ leak decreases SR Ca²⁺ that is necessary for ECC. Additionally, chronic SR Ca²⁺ leak results in mitochondrial calcium overload, which impairs mitochondrial function manifested as oxidative overload and reduced ATP production. SR Ca²⁺ leak can also activate the calcium-activated protease calpain, which can cause cellular injury. The oxidative stress, in turn, can further contribute to RyR1 Ca²⁺ leak by channel oxidation and nitrosylation.

In some embodiments, the musculoskeletal disorder or disease is muscular dystrophy. Non-limiting examples of muscular dystrophy include Duchenne Muscular Dystrophy (DMD), Becker's Muscular Dystrophy (BMD), Limb-Girdle Muscular Dystrophy (LGMD), facioscapulohumeral dystrophy, myotonic muscular dystrophy, congenital muscular dystrophy (CMD), distal muscular dystrophy, Emery-Dreifuss muscular dystrophy, and oculopharyngeal muscular dystrophy.

Duchenne muscular dystrophy (DMD) is one of the leading lethal childhood genetic diseases. Mutations in dystrophin associated with DMD lead to a complete loss of the dystrophin protein, thereby disrupting the link between the subsarcolemma cytoskeleton and the extracellular matrix. This link is essential for protecting and stabilizing the muscle against contraction induced injury. Sarcolemmal instability due to mutations in dystrophin has a cascade effect. One major effect is increased cytosolic Ca²⁺ concentration, which leads to activation of Ca²⁺-dependent proteases (calpains). Another effect is inflammation and elevated iNOS activity, which can cause oxidation/nitrosylation of proteins, lipids, and DNA. DMD muscle pathology is progressive and far exceeds the instability of the sarcolemma. Thus, the pathology is consistent with the instability of the sarcolemma increasing the susceptibility to further injury. Excessive oxidation or nitrosylation of RyR1 can disrupt the interaction of Calstabin1 with the RyR1 complex, leading to RyR1 leakiness and muscle weakness. Treatment with Rycals improves indices of muscle function.

In some embodiments, the musculoskeletal disorder or disease is cancer cachexia, i.e., cancer associated muscle weakness. In some embodiments, the cancer associated muscle weakness is cancer cachexia, for example, due to a cancer having bone metastases. Muscle weakness and muscle atrophy (cachexia) are common paraneoplastic conditions in cancer patients. These conditions cause significant fatigue and dramatically reduce patients' quality of life. In certain cancers, e.g., prostate and breast cancer with bone metastases, RyR1 is oxidized and induced to become leaky. Repairing the leak by administration of Rycal compounds improves muscle function. Non-limiting examples of cancers associated with cachexia that can be treated with a compound herein include breast cancer, prostate cancer, bone cancer, pancreatic cancer, lung cancer, colon cancer, and gastrointestinal cancer. These conditions cause significant fatigue and dramatically reduce patients' quality of life. The present disclosure provides a method for treating, preventing, and reducing a likelihood of developing muscle weakness in a cancer patient, based, for example, on the presence of a modified (e.g., an oxidized state of RyR1), which state induces RyR1 to become leaky. Prevention or reducing a likelihood of occurrence of the leak by administration of Rycal compounds can improve muscle function.

In some embodiments, the musculoskeletal condition or disease is age-related loss of muscle mass and force (sarcopenia). Sarcopenia contributes to disability and increased mortality. RyR1 from aged mice can be oxidized, cysteine-nitrosylated, and depleted of Calstabin1, compared to RyR1 from younger (3-6 months) adults. Treating aged mice with Rycals can stabilize the binding of Calstabin1 to RyR1, reduce intracellular calcium leak, decrease reactive oxygen species (ROS), and enhance tetanic Ca²⁺ release, muscle-specific force, and exercise capacity.

In some embodiments, the compositions of the present disclosure are useful in treating a condition of the pancreas, for example diabetes. In some embodiments, the compositions of the present disclosure are useful in treating Type II diabetes by reducing a likelihood of occurrence of intracellular calcium leak via leaky RyR2. This leak causes mitochondrial calcium overload, and decreased ATP production, which reduces activation of K_{ATP} channels. Reduced activation of the channels blocks depolarization of the plasma membrane. This blocking decreases activation of the plasma membrane voltage-gated calcium channel, which is the primary source of calcium required for insulin secretion.

### Pharmaceutical Compositions

The compounds of the present disclosure can be administered neat or as pharmaceutical compositions for administration to human or animal subjects in a biologically-compatible form suitable for administration in vivo. Subjects can be, for example, elderly adults, adults, adolescents, pre-adolescents, children, toddlers, infants, neonates, and non-human animals. In some embodiments, a subject is a patient.

Compounds of the disclosure are formulated into pharmaceutical compositions for administration to human subjects in a biologically compatible form suitable for administration in vivo. In some embodiments, the present disclosure provides a pharmaceutical composition comprising compounds disclosed herein in admixture with a pharmaceutically-acceptable excipient, diluent and/or carrier. The pharmaceutically-acceptable carrier is preferably acceptable in the sense of being compatible with the other ingredients of the composition and not deleterious to the recipient thereof.

Non-limiting examples of routes of administration include oral, sublingual, buccal, parenteral (intravenous, intramuscular or subcutaneous), transdermal, per- or trans-cutaneous, intranasal, intra-vaginal, rectal, ocular, and respiratory (via inhalation administration). In some embodiments, the compounds are administered directly into the CNS, for example by intralumbar injection or intreventricular infusion of the compounds directly into the cerebrospinal-fluid (CSF), or by intraventricular, intrathecal or interstitial administration. Administration can be to the subject's muscles, for example, the subject's cardiac or skeletal muscles. In some embodiments, the compound is administered to the subject by targeted delivery to cardiac muscle cells via a catheter inserted into the subject's heart. In some embodiments, the compound is orally administered.

Pharmaceutical compositions for solid oral administration include tablets or dragees, sublingual tablets, gastro-resistant tablets, sachets, capsules including gelatin capsules, powders, and granules. Those for liquid oral, nasal, buccal, or ocular administration include emulsions, solutions, suspensions, drops, syrups, and aerosols. The compounds can also be administered as a suspension or solution via drinking water or with food.

Non-limiting examples of pharmaceutically-acceptable excipients or carriers include organic or inorganic materials that are used as materials for pharmaceutical formulations and are incorporated as any one or more of fillers, diluents, binders, disintegrants, buffers (pH adjusting agents), colorants, emulsifiers, flavor-improving agents, gellants, glidants, surfactants (wetting agents), preservatives, solubilizers, stabilizers, suspending agents, sweeteners, tonicity agents, emulsifiers, dispersing agents, swelling agents, retardants, lubricants, absorbents, plasticizers, and viscosity-increasing agents.

Non-limiting examples of pharmaceutically-acceptable fillers/diluents include cellulose derivatives including microcrystalline cellulose, silicified microcrystalline cellulose carboxymethyl cellulose, methyl cellulose, hydroxypropyl cellulose, hydroxypropyl methylcellulose, ethyl cellulose, starches, sugars such as mannitol, sucrose, lactose, sorbitol, dextrins (e.g., maltodextrin), amino-sugars, alginic acid, sodium alginate, and water.

Non-limiting examples of pharmaceutically-acceptable binders include microcrystalline cellulose, gum tragacanth, gum arabic, gelatin, polyvinylpyrrolidone, copovidone, hydroxypropyl methylcellulose, and starch.

Non-limiting examples of pharmaceutically-acceptable disintegrants include roscarmellose sodium, sodium carboxymethyl starch, and crospovidone.

Non-limiting examples of pharmaceutically-acceptable lubricants include stearates such as magnesium stearate or zinc stearate, stearic acid, sodium stearyl fumarate, talc, glyceryl behenate, sodium lauryl sulfate, polyethylene glycol, and hydrogenated vegetable oil.

Non-limiting examples of pharmaceutically-acceptable glidants include colloidal silicon dioxide, talc, tribasic calcium phosphate, calcium silicate, cellulose, magnesium silicate, magnesium trisilicate, starch, magnesium stearate, talc, and mineral oil. Non-limiting examples of moisture barrier agents include stearic acid.

Non-limiting examples of pharmaceutically-acceptable plasticizers include triethyl citrate.

Non-limiting examples of pharmaceutically-acceptable surfactants include sodium laurylsulfate or polysorbates, polyvinyl alcohol (PVA), polyethylene glycols, polyoxyethylene-polyoxypropylene block copolymers known as “poloxamer”, polyglycerin fatty acid esters such as decaglyceryl monolaurate and decaglyceryl monomyristate, sorbitan fatty acid ester such as sorbitan monostearate, polyoxyethylene sorbitan fatty acid ester such as polyoxyethylene sorbitan monooleate (Tween), polyethylene glycol fatty acid ester such as polyoxyethylene monostearate, polyoxyethylene alkyl ether such as polyoxyethylene lauryl ether, polyoxyethylene castor oil, and hardened castor oil such as polyoxyethylene hardened castor oil.

Non-limiting examples of pharmaceutically-acceptable flavoring agents include sweeteners such as sucralose and synthetic flavor oils and flavoring aromatics, natural oils, extracts from plants, leaves, flowers, and fruits, and combinations thereof. Non-limiting examples of flavoring agents include cinnamon oils, oil of wintergreen, peppermint oils, clover oil, hay oil, anise oil, eucalyptus, peppermint, vanilla, citrus oil such as lemon oil, orange oil, grape and grapefruit oil, and fruit essences including apple, peach, pear, strawberry, raspberry, cherry, plum, pineapple, and apricot.

Non-limiting examples of pharmaceutically-acceptable pigments or colorants include alumina (dried aluminum hydroxide), annatto extract, calcium carbonate, canthaxanthin, caramel, p-carotene, cochineal extract, carmine, potassium sodium copper chlorophyllin (chlorophyllin-copper complex), dihydroxyacetone, bismuth oxychloride, synthetic iron oxide, ferric ammonium ferrocyanide, ferric ferrocyanide, chromium hydroxide green, chromium oxide greens, guanine, mica-based pearlescent pigments, pyrophyllite, mica, dentifrices, talc, titanium dioxide, aluminum powder, bronze powder, copper powder, and zinc oxide.

Non-limiting examples of buffering or pH adjusting agents include acidic buffering agents such as short chain fatty acids, citric acid, acetic acid, hydrochloric acid, sulfuric acid and fumaric acid; and basic buffering agents such as tris, sodium carbonate, sodium bicarbonate, sodium hydroxide, potassium hydroxide, and magnesium hydroxide.

Non-limiting examples of tonicity enhancing agents include ionic and non-ionic agents such as, alkali metal or alkaline earth metal halides, urea, glycerol, sorbitol, mannitol, propylene glycol, and dextrose.

Non-limiting examples of wetting agents include glycerin, cetyl alcohol, and glycerol monostearate.

Non-limiting examples of preservatives include benzalkonium chloride, benzoxonium chloride, thiomersal, phenylmercuric nitrate, phenylmercuric acetate, phenylmercuric borate, methylparaben, propylparaben, chlorobutanol, benzyl alcohol, phenyl alcohol, chlorohexidine, and polyhexamethylene biguanide.

Non-limiting examples of antioxidants include sorbic acid, ascorbic acid, ascorbate, glycine, α-tocopherol, butylated hydroxyanisole (BHA), and butylated hydroxytoluene (BHT).

In some embodiments, solid dosage forms are coated. In some embodiments, solid dosage forms contain a core, a subcoating layer substantially surrounding the core, and a coating layer substantially surrounding the subcoating layer.

In some embodiments, the subcoating layer comprises a swellable polymer such as a swellable hydrophobic polymer layer (e.g., hydroxypropyl cellulose (HPC) or hydroxypropylmethyl cellulose (HPMC).

In some embodiments, the coating layer comprises an enteric polymer. Non-limiting examples of enteric polymers include hydroxypropyl methylcellulose acetate succinate (hypromellose acetate succinate, HPMC-AS), cellulose acetate phthalate, hydroxypropyl methylcellulose phthalate, cellulose acetate trimellitate, polyvinyl acetate phthalate, methacrylic acid/methacrylic acid ester copolymers (e.g., poly(methacrylic acid-co-methyl methacrylate), methacrylic acid/acrylic acid ester copolymers, shellac (esters of aleurtic acid).

In some embodiments, pharmaceutically-acceptable carriers or excipients are used to formulate liquids, gels, syrups, elixirs, slurries, or suspensions for oral ingestion by a subject. Non-limiting examples of solvents used in an oral dissolvable formulation can include water, ethanol, isopropanol, saline, physiological saline, DMSO, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, and potassium phosphate buffer.

Pharmaceutical compositions for parenteral injections can include sterile solutions, which can be aqueous or non-aqueous, dispersions, suspensions, emulsions, and also sterile powders for the reconstitution of injectable solutions or dispersions. The compounds can be combined with a sterile aqueous solution that is isotonic with the blood of the subject. A parenteral formulation can be prepared by dissolving a solid active ingredient in water containing physiologically-compatible substances, such as sodium chloride or glycine, and having a buffered pH compatible with physiological conditions, to produce an aqueous solution, then rendering the solution sterile. The formulation is presented in unit or multi-dose containers, such as sealed ampoules or vials. The formulation is delivered by any mode of injection, including, without limitation, epifascial, intracapsular, intracranial, intracutaneous, intrathecal, intramuscular, intraorbital, intraperitoneal, intraspinal, intrasternal, intravascular, intravenous, parenchymatous, subcutaneous, or sublingual or by catheter into the subject's heart.

Pharmaceutical compositions for rectal or vaginal administration can be suppositories, and those for per- or trans-cutaneous administration include powders, aerosols, creams, ointments, gels, and patches.

For transdermal administration, the compounds can be combined with skin penetration enhancers, such as propylene glycol, polyethylene glycol, isopropanol, ethanol, oleic acid, or N-methylpyrrolidone. These agents increase the permeability of the skin and permit compounds to penetrate through the skin and into the bloodstream. The compound/enhancer compositions can be further combined with a polymeric substance, such as ethylcellulose, hydroxypropyl cellulose, ethylene/vinylacetate, or polyvinyl pyrrolidone to provide the composition in gel form, which is dissolved in a solvent, evaporated to the desired viscosity, and then applied to backing material to provide a patch.

Pharmaceutical formulations of the present disclosure can be prepared by methods such as wet granulation, dry granulation, or direct compression.

A pharmaceutically-acceptable excipient can be present in a pharmaceutical composition at a mass of between about 0.1% and about 99% by mass of the composition. For example, a pharmaceutically-acceptable excipient can be present in a pharmaceutical composition at a mass of between about 0.1% and about 95%, between about 0.11% and about 90%, between about 0.1% and about 85%, between about 0.1% and about 80%, between about 0.1% and about 75%, between about 0.1% and about 70%, between about 0.1% and about 65%, between about 0.1% and about 60%, between about 0.1% and about 55%, between about 0.1% and about 50%, between about 0.1% and about 45%, between about 0.11% and about 40%, between about 0.1% and about 35%, between about 0.1% and about 30%, between about 0.1% and about 25%, between about 0.1% and about 20%, between about 0.1% and about 15%, between about 0.1% and about 10%, between about 0.1% and about 5%, between about 0.1% and about 1%, by mass of the formulation.

A pharmaceutically-acceptable excipient can be present at about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% by mass of the formulation.

### Dosing and Dosing Regimens

In accordance with the methods of the present disclosure, any of these compounds can be administered to the subject (or contacted with cells of the subject) in an amount effective to limit or prevent a decrease in the level of RyR-bound Calstabin in the subject, particularly in cells of the subject. Alternatively, the methods of the present disclosure comprise administering a compound in an amount effective to treat or prevent a RyR-related condition as described herein.

In some embodiments, a suitable amount of the compounds effective to limit or prevent a decrease in the level of RyR-bound Calstabin in the subject and/or to treat or prevent conditions associated with RyR ranges from about 1 to about 2,000 mg per day, for example about 10 mg per day, about 20 mg per day, about 30 mg per day, about 40 mg per day, about 50 mg per day, about 60 mg per day, about 70 mg per day, about 80 mg per day, about 90 mg per day, about 100 mg per day, about 120 mg per day, about 140 mg per day, about 160 mg per day, about 180 mg per day, about 200 mg per day, about 220 mg per day, about 240 mg per day, about 260 mg per day, about 280 mg per day, about 300 mg per day, about 320 mg per day, about 340 mg per day, about 360 mg per day, about 380 mg per day, about 400 mg per day, about 420 mg per day, about 440 mg per day, about 460 mg per day, about 480 mg per day, about 500 mg per day, about 600 mg per day, about 700 mg per day, about 800 mg per day, about 900 mg per day, about 1,000 mg per day, about 1,100 mg per day, about 1,200 mg per day, about 1,300 mg per day, about 1,400 mg per day, about 1,500 mg per day, about 1,600 mg per day, about 1,700 mg per day, about 1,800 mg per day, about 1,900 mg per day, or about 2,000 mg per day.

A compound described herein can be present in a composition in a range of from about 1 mg to about 2000 mg; from about 1 mg to about 1000 mg; from about 1 mg to about 500 mg; from about 5 mg to about 1000 mg, from about 5 mg to about 500 mg, from about 5 mg to about 100 mg, from about 10 mg to about 50 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, a dose can be expressed in terms of an amount of the drug divided by the mass of the subject, for example, milligrams of drug per kilograms of subject body mass. In some embodiments, a compound is administered in an amount ranging from about 0.01 mg/kg to about 2,000 mg/kg, about 0.01 mg/kg to about 1,000 mg/kg, about 0.01 mg/kg to about 100 mg/kg, about 0.01 mg/kg to about 10 mg/kg, about 0.01 mg/kg to about 5 mg/kg, about 0.01 mg/kg to about 1 mg/kg, about 0.01 mg/kg to about 0.5 mg/kg, about 0.01 mg/kg to about 0.1 mg/kg, about 0.01 mg/kg to about 0.05 mg/kg, about 1 mg/kg to about 1,000 mg/kg, about 1 mg/kg to about 500 mg/kg, about 1 mg/kg to about 250 mg/kg, about 1 mg/kg to about 100 mg/kg, about 1 mg/kg to about 50 mg/kg, about 5 mg/kg to about 50 mg/kg, about 5 mg/kg to about 10 mg/kg, about 5 mg/kg to about 20 mg/kg, about 10 mg/kg to about 50 mg/kg, about 10 mg/kg to about 20 mg/kg, about 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg. In some embodiments, a compound is administered in an amount of about 1 mg/kg, about 2 mg/kg, about 5 mg/kg, about 10 mg/kg, about 20 mg/kg, about 50 mg/kg, about 100 mg/kg, about 150 mg/kg, about 200 mg/kg, about 250 mg/kg, about 300 mg/kg, about 350 mg/kg, about 400 mg/kg, about 450 mg/kg, about 500 mg/kg, about 550 mg/kg, about 600 mg/kg, about 650 mg/kg, about 700 mg/kg, about 750 mg/kg, about 800 mg/kg, about 850 mg/kg, about 900 mg/kg, about 950 mg/kg or about 1,000 mg/kg of subject body mass.

In some embodiments, a dose can be expressed in terms of an amount of the drug divided by the mass of the subject per day, for example, milligrams of drug per kilograms of subject body mass, per day (mg/kg/day/day). In some embodiments, a compound is administered in an amount ranging from about 0.01 mg/kg/day to about 2,000 mg/kg/day, about 0.01 mg/kg/day to about 1,000 mg/kg/day, about 0.01 mg/kg/day to about 100 mg/kg/day, about 0.01 mg/kg/day to about 10 mg/kg/day, about 0.01 mg/kg to about 5 mg/kg/day, about 0.01 mg/kg/day to about 1 mg/kg/day, about 0.01 mg/kg/day to about 0.5 mg/kg/day, about 0.01 mg/kg/day to about 0.1 mg/kg/day, about 0.01 mg/kg/day to about 0.05 mg/kg/day, about 1 mg/kg/day to about 1,000 mg/kg/day, about 1 mg/kg/day to about 500 mg/kg/day, about 1 mg/kg/day to about 250 mg/kg/day, about 1 mg/kg/day to about 100 mg/kg/day, about 1 mg/kg/day to about 50 mg/kg/day, about 5 mg/kg/day to about 50 mg/kg/day, about 5 mg/kg/day to about 10 mg/kg/day, about 5 mg/kg/day to about 20 mg/kg/day, about 10 mg/kg/day to about 50 mg/kg/day, about 10 mg/kg/day to about 20 mg/kg/day, about 250 mg/kg/day to about 2000 mg/kg/day, about 10 mg/kg/day to about 800 mg/kg/day, about 50 mg/kg/day to about 400 mg/kg/day, about 100 mg/kg/day to about 300 mg/kg/day, or about 150 mg/kg/day to about 200 mg/kg/day. In some embodiments, a compound is administered in an amount of about 1 mg/kg/day, about 2 mg/kg/day, about 5 mg/kg/day, about 10 mg/kg/day, about 20 mg/kg/day, about 50 mg/kg/day, about 100 mg/kg/day, about 150 mg/kg/day, about 200 mg/kg/day, about 250 mg/kg/day, about 300 mg/kg/day, about 350 mg/kg/day, about 400 mg/kg/day, about 450 mg/kg/day, about 500 mg/kg/day, about 550 mg/kg/day, about 600 mg/kg/day, about 650 mg/kg/day, about 700 mg/kg/day, about 750 mg/kg/day, about 800 mg/kg/day, about 850 mg/kg/day, about 900 mg/kg/day, about 950 mg/kg/day or about 1,000 mg/kg/day of subject body mass per day.

In some embodiments, a compound of the disclosure is administered in an amount sufficient to achieve a maximum plasma concentration in a subject (e.g., at steady state) of about 1 ng/ml to about 5,000 ng/ml, for example about 50 ng/ml to about 5,000 ng/ml, about 100 ng/ml to about 5,000 ng/ml, about 200 ng/ml to about 5,000 ng/ml, about 300 ng/ml to about 5,000 ng/ml, about 400 ng/ml to about 5,000 ng/ml, about 500 ng/ml to about 5,000 ng/ml, about 50 ng/ml to about 500 ng/ml, about 100 ng/ml to about 500 ng/ml, about 150 ng/ml to about 500 ng/ml, about 200 ng/ml to about 500 ng/ml, or about 250 ng/ml to about 500 ng/ml.

### Methods of Synthesis

The present disclosure provides processes for the preparation of a compound described herein, or pharmaceutically-acceptable salts thereof. In some embodiments, the present disclosure provides processes for the preparation of compounds of Formula (I). A general route of synthesis) is set forth in Scheme 1:

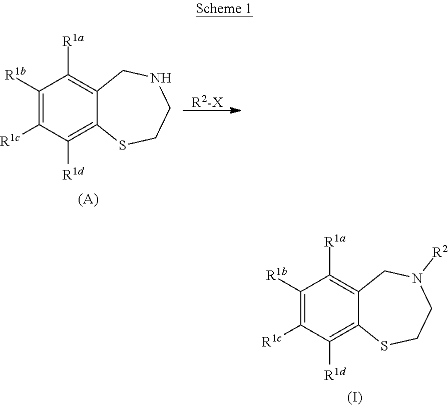

wherein
each R^{1a}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR⁷, each of which is independently substituted or unsubstituted, or hydrogen or halogen;
R² is alkyl, haloalkyl, haloalkoxy, alkenyl, alkynyl, cycloalkyl, aryl, benzyl, heteroaryl, heterocyclyl, —C(O)NR³R⁴, —C(O)C(O)NR³R⁴, —C(O)R⁸, —C(O)OR⁸, or —C(O)C(O)OR⁸, each of which is independently substituted or unsubstituted;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted;
each R⁵, R⁶, R⁷, and R⁸ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; and
X is a leaving group,
   or a pharmaceutically-acceptable salt thereof.

The leaving group X can be, for example, a halogen, a sulfonate (OSO₂)R′ wherein R′ is alkyl or aryl, e.g., OMs (mesylate), OTs (tosylate), imidazole, phenoxy or a substituted phenoxy (e.g., nitrophenoxy, C₆F₅O).

In some embodiments, R² in formula (I) is —NR³R⁴, and the compound is of Formula (I′). In some embodiments, the present disclosure provides processes for the preparation compounds of Formula (I′). A general route of synthesis is set forth in Scheme 2:

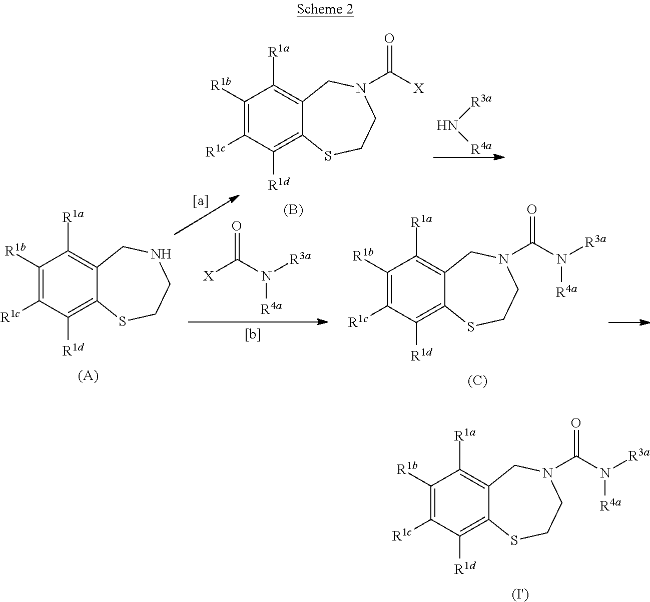

wherein
each R^{1a}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR⁷, each of which is independently substituted or unsubstituted, or hydrogen or halogen;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted;
R^{3a} is R³ or a nitrogen protecting group;
R^{4a} is R⁴ or a nitrogen protecting group;
each R⁵, R⁶, and R⁷ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; and
X is a leaving group as defined in Scheme 1,
   or a pharmaceutically-acceptable salt thereof.

In one embodiment of Scheme 2, (path [a]), the amine starting material (A) is reacted with an acylating reagent such as phosgene, triphosgene, carbonyl diimidazole, thionyl chloride, 4-nitrophenylchloroformate, etc., to yield an acyl intermediate (B), which is reacted with an amine of formula HNR^{3a}R^{4a}, optionally in the presence of a base to yield intermediate (C). The amine HNR³R⁴ optionally comprises a nitrogen protecting group. In alternative path [b], the amine starting material (A) is reacted with an acylated amine X—C(O)—NR^{3a}R^{4a}, to yield intermediate (C). The nitrogen protecting group, if present, can be removed to yield a compound of formula (I′).

In some embodiments, R² in formula (I) is piperazinyl or a substituted piperazinyl, and the compound is of Formula (II). In some embodiments, the present disclosure provides processes for the preparation compounds of Formula (II). A general route of synthesis is set forth in Scheme 3:

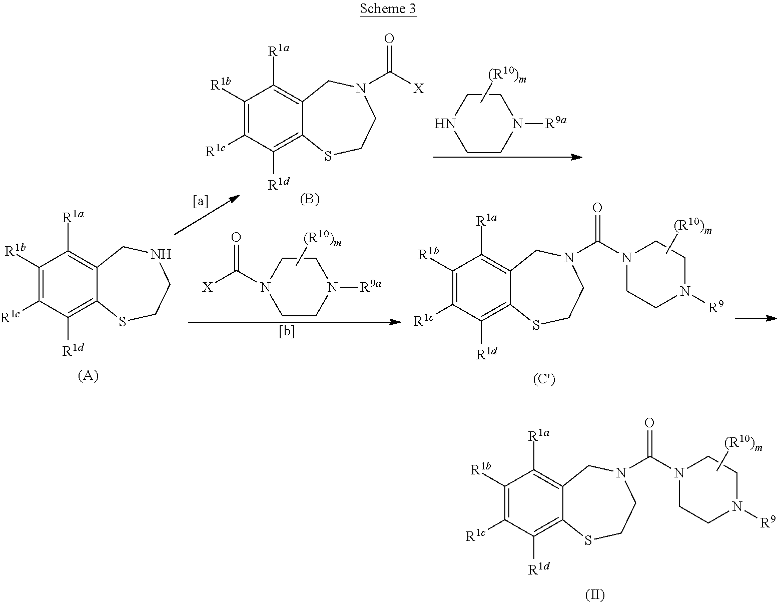

wherein
each R^{1a}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR⁷, each of which is independently substituted or unsubstituted, or hydrogen or halogen;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted;
each R⁵, R⁶, and R⁷ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
R⁹ is alkyl, alkenyl, alkynyl, cycloalkyl, aryl, benzyl, heterocyclyl, heteroaryl, —C(O)NR³R⁴, —C(O)R⁸, or —C(O)OR⁸, each of which is independently substituted or unsubstituted, or hydrogen;
R^{9a} is R⁹ or a nitrogen protecting group;
each R¹⁰ is independently alkyl, alkenyl, alkynyl, cycloalkyl, aryl, benzyl, heterocyclyl, heteroaryl, —NR³R⁴, —OR⁵, or —SR⁷, each of which is unsubstituted or substituted;
m is 0, 1, 2, 3, 4, 5, 6, 7, or 8; and
X is a leaving group as defined in Scheme 1,
   or a pharmaceutically-acceptable salt thereof.

In some embodiments of Scheme 3 (path [a]), the amine starting material (A) is reacted with an acylating reagent such as phosgene, triphosgene, carbonyl diimidazole, thionyl chloride, 4-nitrophenylchloroformate, etc., to yield an acyl intermediate (B), which is reacted with piperazine (R^{9a}═H), a substituted piperazine (R^{9a} is other than H), or a protected derivative thereof (R^{9a}=PG, a nitrogen protecting group), optionally in the presence of a base to yield intermediate (C′). In alternative path [b], the amine starting material (A) is reacted with an acylated piperazine, or a substituted acylated piperazine, or a protected acylated piperazine derivative to directly yield intermediate (C′). The protecting group, if present, can be removed to yield a compound of formula (II).

In some embodiments, the compound is formula (III), and the route of synthesis is described in Scheme 3a:

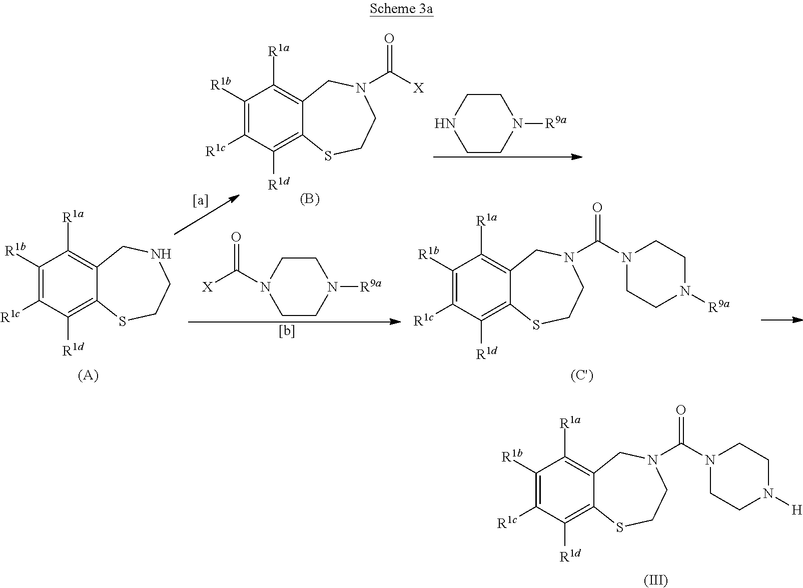

wherein
each R^{1a}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR⁷, each of which is independently substituted or unsubstituted, or hydrogen or halogen;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted;
each R⁵, R⁶, and R⁷ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
R^{9a} is hydrogen or a nitrogen protecting group; and
X is a leaving group as defined in Scheme 1,
   or a pharmaceutically-acceptable salt thereof.

In some embodiments of Scheme 3a (path [a]), the amine starting material (A) is reacted with an acylating reagent such as phosgene, triphosgene, carbonyl diimidazole, thionyl chloride, 4-nitrophenylchloroformate, etc., to yield an acyl intermediate (B), which is reacted with piperazine (R^{9a}=H) or a protected derivative thereof (R^{9a}=PG, a nitrogen protecting group), optionally in the presence of a base to yield intermediate (C′). In alternative path [b], the amine starting material (A) is reacted with an acylated piperazine or a protected acylated piperazine derivative to directly yield intermediate (C′). The protecting group, if present, can be removed to yield a compound of formula (III).

### Preparation of Amine Stating Material (A)

The amine starting material (A) can generally be prepared as depicted in Scheme 4, (Method 1), Scheme 5 (Method 2) or Scheme 6 (Method 3):

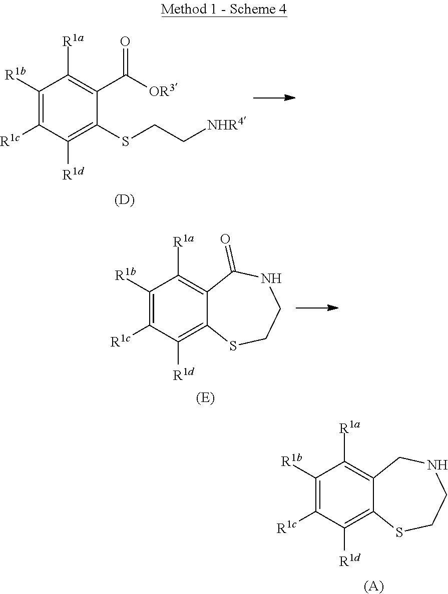

In Scheme 4, R^{1a}, R^{1b}, R^{1c}, and R^{1d} are as defined above, R^{3′} is alkyl or aryl, and R^{4′} is H or PG, wherein PG is a nitrogen protecting group. The starting material (D) is cyclized, optionally in the presence of a base, to yield the corresponding benzothiazepanone (E), which is reduced to yield compound (A) or a salt thereof, e.g., a hydrochloride salt or a hydrobromide salt. Any of the bases described herein can be used for this purpose. The amine group in starting material (D) can optionally be protected, in which case the protecting group is removed prior to cyclization. The starting material (D) can be in the form of a free amine or in the form of an acid addition salt, e.g., a hydrochloride salt or a hydrobromide salt.

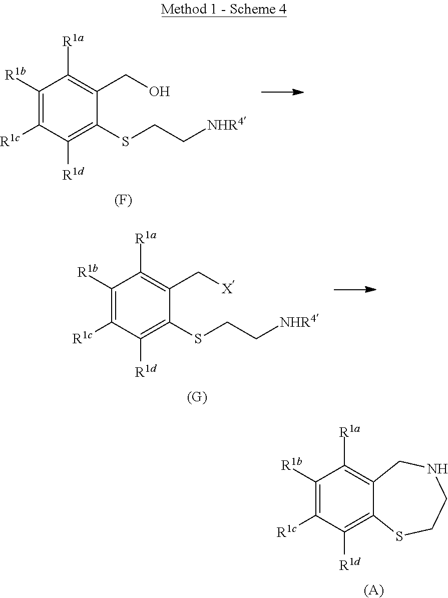

In Scheme 5, R^{1a}, R^{1b}, R^{1c}, and R^{1d} are as defined above, X′ is a leaving group as defined above for X, and R^{4′} is H or PG, wherein PG is a nitrogen protecting group. The starting material alcohol (F) is reacted with an activating agent to introduce the group X′, followed by cyclization of the intermediate (G), optionally in the presence of a base, to yield the corresponding benzothiazepine derivative of formula (A) or a salt thereof, e.g., a hydrochloride salt or a hydrobromide salt. Any of the bases described herein can be used for this purpose. The amine group in starting material (G) can optionally be protected, in which case the protecting group is removed prior to cyclization. The starting material (F) and/or the intermediate (G) can be in the form of a free amine or in the form of an acid addition salt, e.g., a hydrochloride salt or a hydrobromide salt.

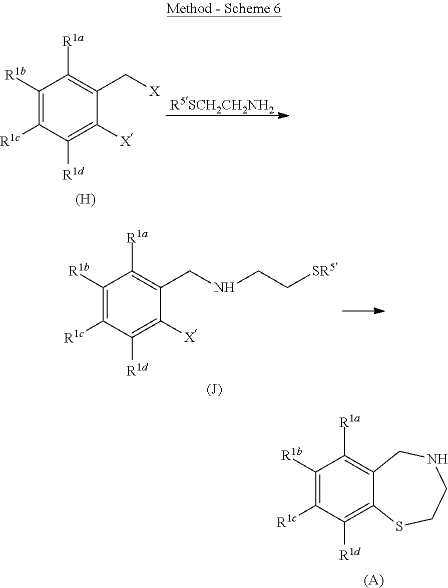

In Scheme 6, R^{1a}, R^{1b}, R^{1c} and R^{1d} are as defined above, X and X′ are each a leaving group as defined above, and R^{5′} is H or PG′, wherein PG′ is a sulfur protecting group (e.g., trityl). Alternatively, the S—S dimer (S—CH₂CH₂NH₂)₂ can be used in lieu of R^{5′}S—CH₂—CH₂—NH₂. The starting material (H) is coupled with an optionally protected 2-aminoethanthiol to yield intermediate (J), followed by (optional) deprotection and cyclization to yield the corresponding benzothiazepine derivative of formula (A) or a salt thereof, e.g., a hydrochloride salt or a hydrobromide salt. The intermediate (J) can be in the form of a free amine or in the form of an acid addition salt, e.g., a hydrochloride salt or a hydrobromide salt.

### Alternative Synthetic Embodiments

In some embodiments, the present disclosure provides processes for the preparation compounds of Formula (IV). A general route of synthesis (ROS) is set forth in Scheme 7:

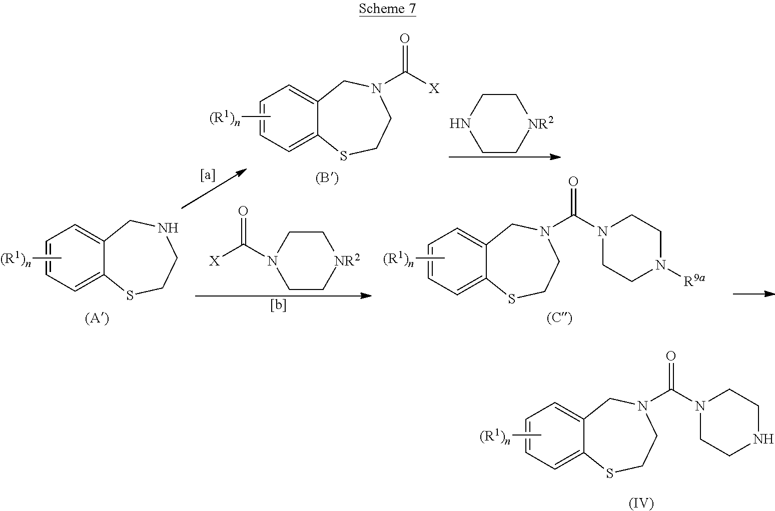

wherein
R¹ is halogen, haloalkyl or haloalkyloxy;
n is 1, 2, 3, or 4;
R² is hydrogen or PG, wherein PG is a nitrogen protecting group; and
X is a leaving group as defined in Scheme 1.

In one embodiment of Scheme 7 (path [a]), the amine starting material (A′) is reacted with an acylating reagent such as phosgene, triphosgene, carbonyl diimidazole, thionyl chloride, 4-nitrophenylchloroformate, etc., to yield an acyl intermediate (B′), which is reacted with piperazine (R²=H) or a protected derivative thereof (R²=PG, a nitrogen protecting group) optionally in the presence of a base to yield intermediate (C″). In alternative path [b], the amine starting material (A′) is reacted with an acylated piperazine derivative to yield intermediate (C″). The protecting group, if present, is then removed to yield a compound of formula (IV).

### Preparation of Starting Material (A′)

The amine starting material (A′) can generally be prepared as depicted in Scheme 8, (Method 1′), Scheme 9 (Method 2′) or Scheme 10 (Method 3′), as described above for the preparation of compound (A).

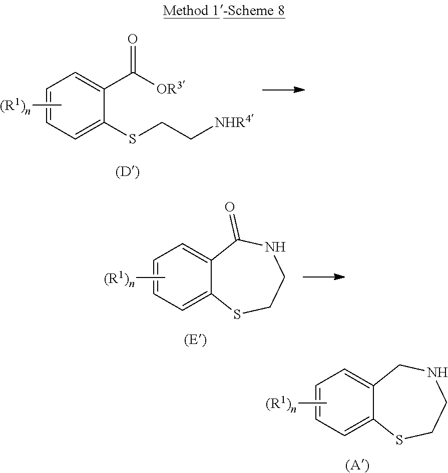

In Scheme 8, R¹ and n are as defined above, R^{3′} is alkyl or aryl, and R^{4′} is H or PG, wherein PG is a nitrogen protecting group. The starting material (D′) is cyclized, optionally in the presence of a base, to yield the corresponding benzothiazepanone (E′), which is reduced to yield compound (A′) or a salt thereof, e.g., a hydrochloride salt or a hydrobromide salt. Any of the bases described herein can be used for this purpose. The amine group in starting material (D′) can optionally be protected, in which case the protecting group is removed prior to cyclization. The starting material (D′) can be in the form of a free amine or in the form of an acid addition salt, e.g., a hydrochloride salt or a hydrobromide salt.

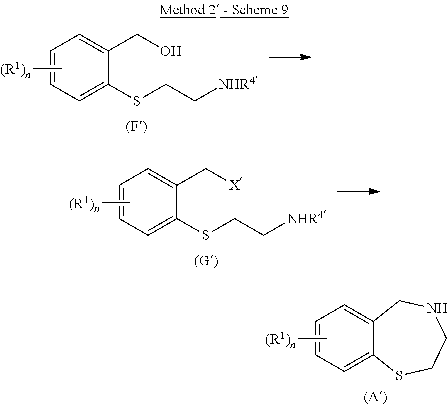

In Scheme 9, R¹ and n are as defined above, X′ is a leaving group as defined above for X, and R^{4′} is H or PG, wherein PG is a nitrogen protecting group. The starting material alcohol (F′) is reacted with an activating agent to introduce the group X′, followed by cyclization of the intermediate (G′), optionally in the presence of a base, to yield the corresponding benzothiazepine derivative of formula (A′) or a salt thereof, e.g., a hydrochloride salt or a hydrobromide salt. Any of the bases described herein can be used for this purpose. The amine group in starting material (G′) can optionally be protected, in which case the protecting group is removed prior to cyclization. The starting material (F′) and/or the intermediate (G′) can be in the form of a free amine or in the form of an acid addition salt, e.g., a hydrochloride salt or a hydrobromide salt.

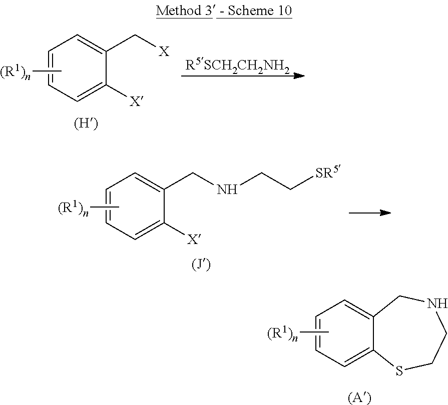

In Scheme 10, R¹ and n are as defined above, X and X′ are each a leaving group as defined above, and R⁵ is H or PG′, wherein PG′ is a sulfur protecting group (e.g., trityl). Alternatively, the S—S dimer (S—CH₂CH₂NH₂)₂ could be used in lieu of R⁵'S—CH₂—CH₂—NH₂. The starting material (H′) is coupled with an optionally protected 2-aminoethanthiol to yield intermediate (J′), followed by (optional) deprotection and cyclization to yield the corresponding benzothiazepine derivative of formula (A′) or a salt thereof, e.g., a hydrochloride salt or a hydrobromide salt. The intermediate (J) can be in the form of a free amine or in the form of an acid addition salt, e.g., a hydrochloride salt or a hydrobromide salt.

In some embodiments, the compound of formula (I), (I″) (II), (III), or (IV) can be converted into a pharmaceutically acceptable salt thereof, for example, a salt with a pharmaceutically-acceptable acid. Salts of compounds of formula (I), (I′) (II), (III), or (IV) can be prepared by reacting the parent molecule with a suitable acid (e.g., hydrobromic acid, hydrofluoric acid, trifluoroacetic acid, sulfuric acid, phosphoric acid, acetic acid, succinic acid, citric acid, lactic acid, maleic acid, fumaric acid, palmitic acid, cholic acid, pamoic acid, mucic acid, D-glutamic acid, D-camphoric acid, glutaric acid, phthalic acid, tartaric acid, lauric acid, stearic acid, salicyclic acid, methanesulfonic acid, benzenesulfonic acid, sorbic acid, picric acid, benzoic acid, or cinnamic acid). In some embodiments, the salt is a hydrochloric acid salt. In some embodiments, the compounds can also be isolated directly as salts, without proceeding through the free amine base. This result can be achieved, for example, by removing the protecting group with an acid that directly forms an acid addition salt with the compound of formula (I), (I′) (II), (III), or (IV). Non-limiting examples of suitable acids are as described above.

The nature of the base used in reactions described herein is not limiting. Non-limiting examples of bases include an organic base such as a tertiary amine, including acyclic amines (e.g., trimethylamine, triethylamine, N,N-dimethylphenylamine N,N-diisopropylethylamine (DIEA) and tributylamine), cyclic amines (e.g., N-methylmorpholine) and aromatic amines (dimethylaniline, dimethylaminopyridine and pyridine).

A protecting group can mask a functionality during a process step in which the functionality would otherwise react in an undesirable way. The protecting group is subsequently removed to expose the original functionality. The removal or deprotection occurs after the completion of the reaction or reactions in which the functionality would interfere.

In some embodiments, a functional group to be protected is an amine group. Suitable protecting groups include, for example, groups of the formula —C(═O)—R; wherein R is (C₁-C₄) alkoxy, allyloxy, benzyloxy, substituted benzyloxy, fluorenylmethoxy or adamantyloxy. Non-limiting examples of nitrogen protecting groups are t-butoxycarbonyl (BOC), benzyloxycarbonyl, substituted benzyloxycarbonyl or fluorenylmethoxycarbonyl (FMOC). Additional nitrogen protecting groups include trityl, acyl (e.g., trifluoroacetyl), alkylaryl (e.g., benzyl), SOS₂R′ wherein R′ is alkyl or aryl, e.g., OMs (mesylate), and OTs (tosylate)).

Other suitable protecting groups are discussed in standard textbooks in the field of chemistry, such as Protective Groups in Organic Synthesis by T. W. Greene and P. G. M. Wuts [John Wiley & Sons, New York, 1999], which is incorporated herein by reference in its entirety as if fully set forth herein. Particular attention is drawn to the chapter titled “Protection for the Amino Group” (pages 494-614).

The reaction can be conducted in the presence or absence of a solvent. The nature of the solvent, when used, is not limiting, with examples including solvents such an ester (e.g., ethyl acetate), an ether (e.g., THF), a chlorinated solvent (e.g., dichloromethane or chloroform), dimethylformamide (DMF), and other solvents such as acetonitrile or toluene or mixtures of these solvents with each other or with water.

### EXAMPLES

### Example 1: General Synthetic Methods

Instruments:
NMR: Bruker AVANCE 111400 or Varian Mercury 300
LC/MS: Waters Delta 600 equipped with Autosampler 717Plus, Photo Diode—Array Detector 2996, and Mass Detector 3100, or Shimadzu 210

### Ex. 1A. General Procedure for the Preparation of Substituted 2,3,4,5-tetrahydrobenzo[f][1,4]thiazepine (“Amine”, Compound A) (Method 1, Scheme 4)

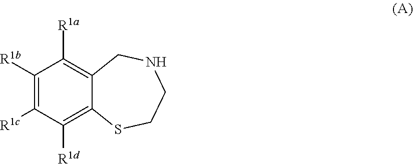

To a stirred solution of Boc-protected ester (D) (100 mmol) in organic solvent was added TFA or HCl (excess). The solution was stirred at room temperature and the TFA and organic solvents were removed. The residue was dissolved in organic solvent and sodium methoxide (200 mmol) was added. The reaction mixture was stirred overnight at RT followed by removal of solvents under reduced pressure. The residue was washed with water/acid and the precipitate was collected and dried, to afford pure lactam (E).

To a stirred solution of lactam (E) was in anhydrous organic solvent was added a reducing agent such as borane tetrahydrofuran (BH₃:THF) or lithium aluminum hydride (2 eq.) at 0° C. The reaction mixture was heated to reflux, stirred overnight, then quenched with methanol. The mixture was refluxed for 1 hr and the solvent was removed by evaporation. The residue was suspended in ethanol and treated with concentrated HCl followed by removal of solvent. The product was isolated by removing the unreacted starting material by precipitation and filtration from an acidic aqueous solution, followed by additional organic extraction. The aqueous solution was made basic and the product was obtained by organic extraction.

### Ex. 1B. General Procedure for the Preparation of Substituted 2,3,4,5-tetrahydrobenzo[f][1,4]thiazepine (“Amine”, Compound A) (Method 2, Scheme 5)

To a stirred solution of Boc-protected (F) was added thionyl chloride dropwise in organic solvent. The reaction mixture was stirred at 0° C. then warmed up to RT. The reaction was quenched with alcohol. After removal of solvents, the desired crude product (G) was obtained.

To a stirred solution of compound (G) obtained above in organic solvent, was added base such as DIEA or i-Pr₂Et, and the reaction was stirred at RT overnight. After the solvents were removed, the residue was purified by column chromatography on silica gel to yield the product as a white solid.

### Ex. 1C. General Procedure for the Preparation of Substituted 2,3,4,5-tetrahydrobenzo[f][1,4]thiazepine (“Amine”, Compound A) (Method 3, Scheme 6)

To a stirred solution of benzyl derivative (H) was added trityl protected cystamine in the presence of a base in organic solvent and the reaction was stirred at RT. The solvent was removed and the residue was purified by column chromatography to yield intermediate (J).

Intermediate (J) was dissolved in organic solvent and the trityl group was removed with TFA/Et₃SiH followed by removal of solvent and TFA. The residue was mixed with CuI with base in alcohol, and the reaction mixture was refluxed overnight, cooled to RT, followed by isolation of the product by filtration.

### Ex. 1D. General Procedure for the Amidation of Substituted 2,3,4,5-tetrahydrobenzo[f][1,4]thiazepine (Amine A) (Schemes 3, 3a)

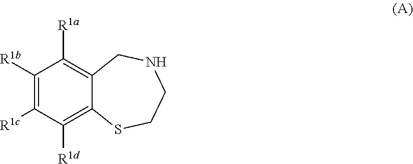

Path [a]: Amine (A) (1 mmol) was dissolved in an organic solvent. To the solution was added acylating reagent (1 mmol), followed by a base (2 mmol). The mixture was stirred at room temperature for 0.5-1 hr. Then, the reaction was quenched in an aqueous solution at 0° C. The organic phase was separated and dried over anhydrous sodium sulfate. Following evaporation of solvent an optional purification, the intermediate was reacted with Boc-piperazine (2 mmol) and base (2.5 mmol) in an organic solvent. The reaction mixture was stirred at 0° C. for 0.5-1 hr and at room temperature overnight. The mixture was washed with aqueous solution and after being dried over anhydrous sodium sulfate, the solution was filtered and concentrated. The residue was purified by column chromatography on silica gel.

Path [b]: Alternatively, amine (A) was dissolved in organic solvent and Boc-protected chlorocarbonyl-piperazine was mixed in. The solution was stirred at room temperature for 24 hours and the reaction solution was evaporated to dryness. The Boc protecting group was removed with TFA. The residue was purified by column chromatography on silica gel.

### Example 2: Preparation of Piperazin-1-yl(8-(trifluoromethyl)-2,3-dihydrobenzo[f][1,4]thiazepin-4(5H)-yl) methanone hydrochloride (Compound 1·HCl)

Compound (1) was prepared according to Scheme (3) or (3a) path [a], and Method 1 (Scheme 4):

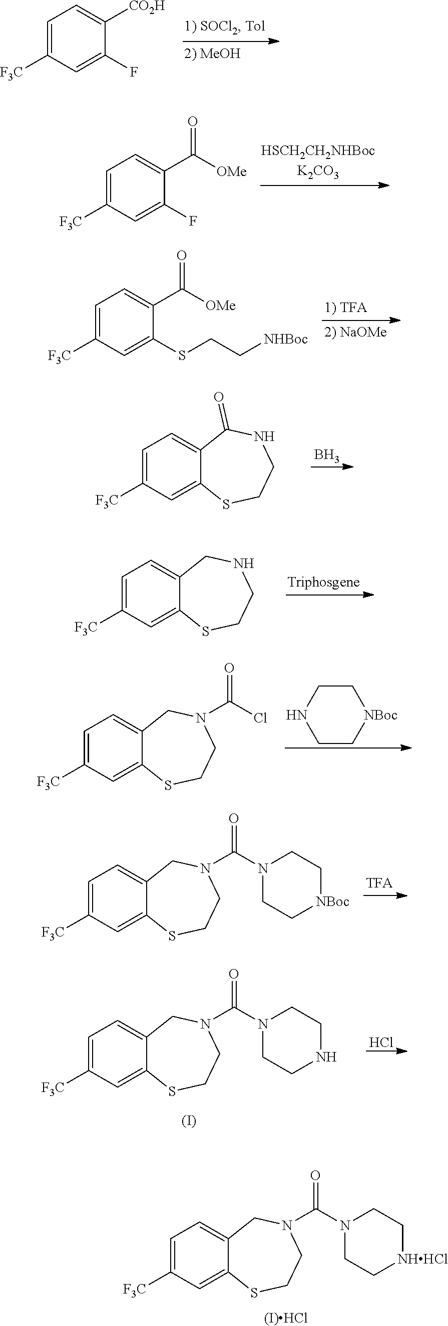

### i) Methyl 2-fluoro-4-(trifluoromethyl)benzoate

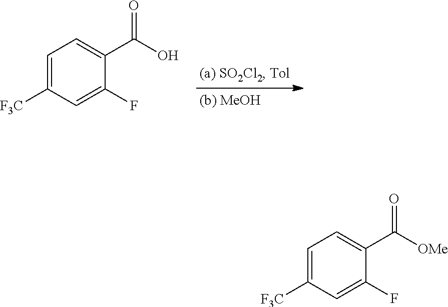 [SMILES: O=C(O)c1ccc(C(F)(F)F)cc1F ; COC(=O)c1ccc(C(F)(F)F)cc1F]

To a stirred solution of 2-fluoro-4-(trifluoromethyl)benzoic acid (24.0 g, 115.3 mmol) in anhydrous toluene (100 ml) was added thionyl chloride (20 ml, 32.7 g, 275 mmol, 2.4 eq) and anhydrous DMF (5 drops). The reaction mixture was refluxed for 3 hrs. After the solvents were removed under reduced pressure, the residue was co-evaporated with toluene (30 ml) once. Then, the residue was dissolved in anhydrous CH₂Cl₂ (100 ml). This solution was then slowly added to anhydrous methanol (100 ml) at 0° C. with stirring. The resulting mixture was stirred at room temperature over weekend. After removal of solvents, a colorless oil crude product was obtained. The crude product was essentially pure. No further purification was attempted.

¹H-NMR (300 MHz, CDCl₃): 8.07 (1, 1H), 7.45 (m, 2H), 3.97 (s, 3H).

### ii) Methyl 2-(2-(tert-butoxycarbonylamino)ethylthio)-4-(trifluoromethyl)benzoate

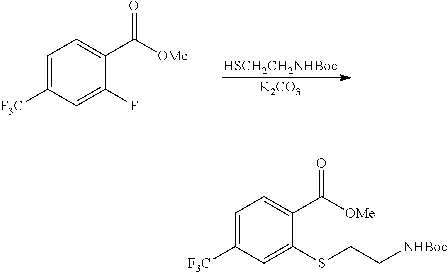 [SMILES: COC(=O)c1ccc(C(F)(F)F)cc1F ; "K2CO3" ; COC(=O)c1ccc(C(F)(F)F)cc1SCCNC(=O)OC(C)(C)C ; "HSCH2CH2NHBoc"]

To a stirred solution of methyl 2-fluoro-4-(trifluoromethyl)benzoate (1.00 g, 4.50 mmol) in anhydrous MeCN (10 ml) was added 2-(Boc-amino)ethanethiol (0.80 ml, 839.2 mg, 4.73 mmol, 1.05 eq) and K₂CO₃ (1.86 g, 13.47 mmol, 3 eq). The mixture was degassed and refilled with Ar for 3 times and refluxed overnight. Then, the solvent was removed under reduced pressure. The residue was dissolved in CH₂Cl₂ (100 ml) and H₂O (100 ml). The organic phase was separated. The aqueous phase was extracted with CH₂Cl₂ (100 ml). The combined CH₂Cl₂ phase was dried over anhydrous sodium sulfate and filtered. Removal of solvent gave a colorless oil, which turned to a white solid after being dried on high vacuum. The product was used in the next step without further purification.

¹H-NMR (300 MHz, CDCl₃): 8.05 (d, 1H), 7.61 (s, 1H), 7.42 (d, 1H), 4.98 (br. S, 1H), 3.95 (s, 3H), 3.46 (q, 2H, q), 3.15 (t, 2H), 1.44 (s, 9H).

### iii) 8-(Trifluoromethyl)-3,4-dihydrobenzo[f][1,4]thiazepin-5(2H)-one

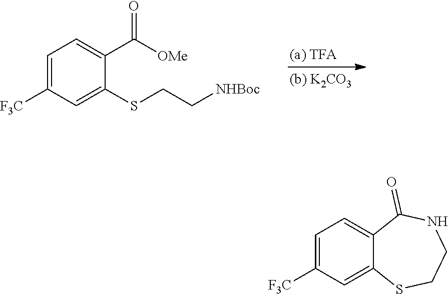 [SMILES: COC(=O)c1ccc(C(F)(F)F)cc1SCCNC(=O)OC(C)(C)C ; O=C1NCCSc2cc(C(F)(F)F)ccc21]

To a stirred solution of methyl 2-(2-(tert-butoxycarbonylamino)ethylthio)-4-(trifluoromethyl)benzoate (1.60 g, 4.22 mmol) in CH₂Cl₂ (5 ml) was added trifluoroacetic acid (5 ml). The mixture was stirred at room temperature for 2 hrs. Removal of solvents under reduced pressure gave to an oil product, which became to a white solid after being dried on high vacuum for 2 hrs.

The above white solid crude product was dissolved in anhydrous MeOH (20 ml) and was treated with a solution of NaOMe in MeOH (4.375 M, 1.93 ml, 8.44 mmol) at room temperature overnight. After the solvent was removed, the residue was treated with 1 M citric acid/H₂O at room temperature for 0.5 hr. The mixture was filtered, and the white solid was washed with H₂O (10 ml×3) and dried on high vacuum. 0.85 g pure white solid product was obtained.

¹H-NMR (300 MHz, CDCl₃): 7.82-7.80 (m, 2H), 7.70-7.67 (m, 1H), 7.09 (br. S, 1H), 3.41 (q, 2H, q), 3.22 (t, 2H).

### iv. 8-(Trifluoromethyl)-2,3,4,5-tetrahydrobenzo[f][1,4]thiazepine

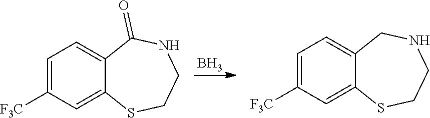

To a stirred solution of 8-(trifluoromethyl)-3,4-dihydrobenzo[f][1,4]thiazepin-5(2H)-one (18.69 g, 75.67 mmol) in anhydrous THF (200 ml) at 0° C. was added dropwise a borane tetrahydrofuran complex solution (1M in THF, 151.3 ml, 151.3 mmol, 2 eq). The reaction mixture was heated to reflux overnight. Then, MeOH (100 ml) was slowly added to the mixture at 0° C., and the mixture was heated to reflux for 1 hr. After the solvents were removed, the oil residue was suspended in MeOH (200 ml) and treated with concentrated HCl (40 ml). After 2 days at reflux, the mixture was still a cloudy solution. Then, the solvents were removed under reduced pressure. The residue was suspended in EtOH (150 ml) and treated with concentrated HCl (30 ml) for 24 hrs until the solution was clear. After removal of solvents, the residue was treated with H₂O (250 ml). Filtration of the cloudy mixture gave to a white solid, which was the unreacted starting material. The filtrate was washed with EtOAc (50 ml, 80 ml and 100 ml). The combined EtOAc phase was dried over anhydrous sodium sulfate and filtered. Removal of solvent gave a white solid compound, which was the unreacted starting material (Total starting material recovered, 5.50 g, 29%). The aqueous phase was treated with NaOH/H₂O to pH=14. The product was extracted with CH₂Cl₂ (100 ml×3). The combined CH₂Cl₂ phase was dried over anhydrous sodium sulfate and filtered. Removal of solvent gave the desired product as colorless oil.

¹H-NMR (300 MHz, CDCl₃): 7.82 (s, 1H), 7.46-7.42 (m, 1H), 7.34-7.31 (m, 1H), 4.17 (s, 2H), 3.42-3.38 (m, 2H), 2.81-2.78 (m, 2H).

### v. 8-(trifluoromethyl)-2,3-dihydrobenzo[f][1,4]thiazepine-4(5H)-carbonyl chloride

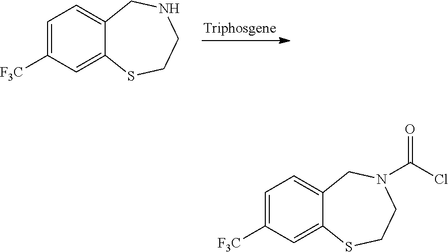 [SMILES: FC(F)(F)c1ccc2c(c1)SCCNC2 ; O=C(Cl)N1CCSc2cc(C(F)(F)F)ccc2C1]

To a stirred solution of triphosgene (300 mg, 1.01 mmol) in CH₂Cl₂ at 0° C. was added slowly in 10 min a solution of 8-(trifluoromethyl)-2,3,4,5-tetrahydrobenzo[f][1,4]thiazepine (300 mg, 1.29 mmol) and pyridine (0.83 ml, 808 mg, 101.1 mmol) in CH₂Cl₂ (5 ml). The reaction mixture was stirred at 0° C. for 30 min. Then, the reaction was quenched by addition of a 0.5 N HCl/H₂O solution (20 ml) at 0° C. The CH₂Cl₂ phase was separated and dried over anhydrous sodium sulfate. After filtration and removal of solvent, the residue was purified by column chromatography on silica gel. The product 8-(trifluoromethyl)-2,3-dihydrobenzo[f][1,4]thiazepine-4(5H)-carbonyl chloride was obtained as colorless oil, 0.32 g.

### vi. tert-butyl 4-(8-(trifluoromethyl)-2,3,4,5-tetrahydrobenzo[f][1,4]thiazepine-4-carbonyl)piperazine-1-carboxylate

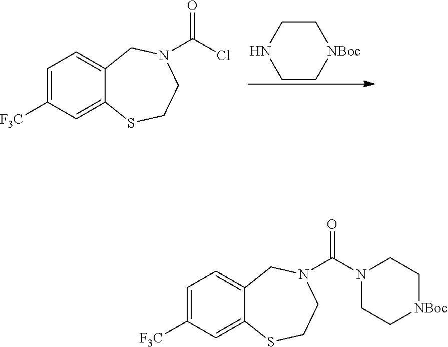

To a stirred solution of Boc-piperazine (360 mg, 1.93 mmol) and pyridine (0.21 ml, 199 mg, 2.49 mmol) in CH₂Cl₂ (10 ml) at 0° C. was added a solution of 8-(trifluoromethyl)-2,3-dihydrobenzo[f][1,4]thiazepine-4(5H)-carbonyl chloride (the product from above step, 0.32 g, 1.08 mmol) in CH₂Cl₂ (10 ml). The reaction mixture was stirred at 0° C. for 0.5 hr and at room temperature overnight. After dilution with CH₂Cl₂ (50 ml), the mixture was washed with saturated NaHCO₃/H₂O (20 ml), 1 M citric acid/H₂O (20 ml×2), and H₂O (20 ml). After being dried over anhydrous sodium sulfate, the solution was filtered and concentrated. The residue was purified by column chromatography on silica gel. The product tert-butyl 4-(8-(trifluoromethyl)-2,3,4,5-tetrahydrobenzo[f][1,4]thiazepine-4-carbonyl)piperazine-1-carboxylate was obtained as colorless oil.

¹H-NMR (300 MHz, CDCl₃): 7.79 (s, 1H), 7.50-7.40 (m, 2H), 4.97 (s, 2H), 3.99 (m, 2H), 3.42 (m, 4H), 3.18 (m, 4H), 2.95 (m, 2H), 1.46 (s, 9H).

### vii. piperazin-1-yl(8-(trifluoromethyl)-2,3-dihydrobenzo[f][1,4]thiazepin-4(5H)-yl)methanone (1·HCl)

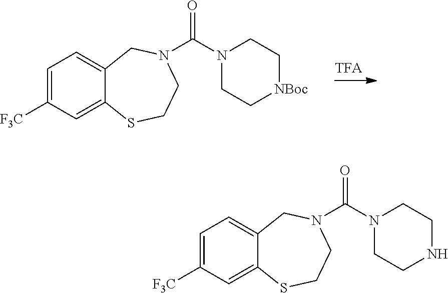 [SMILES: CC(C)(C)OC(=O)N1CCN(C(=O)N2CCSc3cc(C(F)(F)F)ccc3C2)CC1 ; O=C(N1CCNCC1)N1CCSc2cc(C(F)(F)F)ccc2C1]

To a stirred solution of tert-butyl 4-(8-(trifluoromethyl)-2,3,4,5-tetrahydrobenzo[f][1,4]thiazepine-4-carbonyl)piperazine-1-carboxylate (the product obtained from above step, 0.46 g, 1.03 mmol) in CH₂Cl₂ (5 ml) was added trifluoroacetic acid (5 ml). The mixture was stirred at room temperature for 5 hrs. After the solvents were removed under reduced pressure, the residue was dissolved in H₂O (50 ml) and washed with ether (25 ml×2). The aqueous phase was treated with NaOH/H₂O to pH=12. The product was extracted with CH₂Cl₂ (30 ml×3). The combined organic phase was dried over anhydrous sodium sulfate and filtered. Removal of solvent gave the desired product, piperazin-1-yl(8-(trifluoromethyl)-2,3-dihydrobenzo[f][1,4]thiazepin-4(5H)-yl)methanone (1), as white solid.

### viii. piperazin-1-yl(8-(trifluoromethyl)-2,3-dihydrobenzo[f][1,4]thiazepin-4(5H)-yl)methanone (1·HCl)

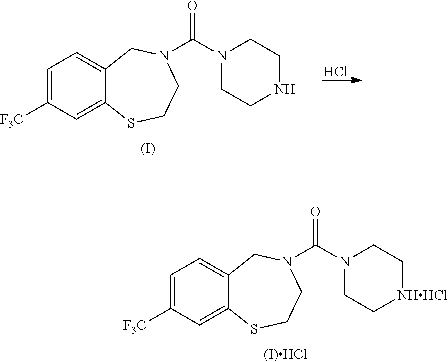

To a stirred solution of piperazin-1-yl(8-(trifluoromethyl)-2,3-dihydro benzo[f][1,4]thiazepin-4(5H)-yl)methanone (1) (0.36 g, 1.04 mmol) in ether (20 ml) was added a 1 M HCl/ether solution (2 ml, 2 mmol). The mixture was stirred at room temperature for 0.5 hr. The solvents were removed. The residue was washed with ether once (1 ml) and dried on high vacuum. The desire product piperazin-1-yl(8-(trifluoromethyl)-2,3-dihydrobenzo[f][1,4]thiazepin-4(5H)-yl)methanone hydrochloride (1·HCl) was obtained as yellow solid.

¹H-NMR (300 MHz, DMSO-d₆): 8.86 (br. S, 2H), 7.69 (1H, s), 7.57 (s, 2H), 4.56 (s, 2H), 3.71 (m, 2H), 3.24 (m, 4H), 3.09 (m, 6H).

### Example 3: Preparation of piperazin-1-yl(7-(trifluoromethyl)-2,3-dihydrobenzo[f][1,4]thiazepin-4(5H)-yl) methanone hydrochloride (Compound 2·HCl)

Compound (2) was prepared according to Scheme (3) or (3a) path [b] and Method 1 (Scheme 4), using the general synthetic methodology described in Example 1.

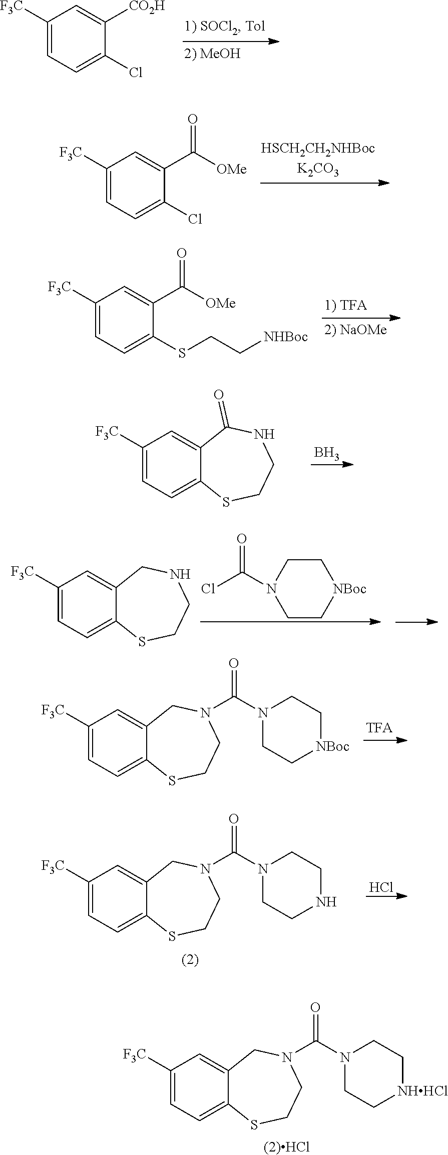

### i) Methyl 5-Trifluoro-2-chloro benzoic acid

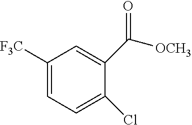 [SMILES: COC(=O)c1cc(C(F)(F)F)ccc1Cl]

¹HNMR (300 MHz, CDCl₃): 8.05 (d, 1H), 7.63 (dd, 1H), 7.56 (d, 1H), 3.97 (s, 3H).

### iii) methyl 2-((2-((tert-butoxycarbonyl)amino)ethyl)thio)-5-(trifluoromethyl)benzoate

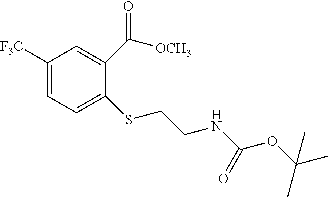 [SMILES: COC(=O)c1cc(C(F)(F)F)ccc1SCCNC(=O)OC(C)(C)C]

¹HNMR (300 MHz, CDCl₃): 8.21 (d, 1H), 7.63 (d, 1H), 7.56 (d, 1H), 4.98 (br s, 1H), 3.97 (s, 3H), 3.44 (q, 2H), 3.10 (1, 2H), 1.45 (s, 9H).

### iii) 7-(Trifluoromethyl)-3,4-dihydrobenzo[f][1,4]thiazepin-5(2H)-one

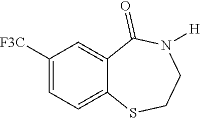 [SMILES: O=C1NCCSc2ccc(C(F)(F)F)cc21]

¹HNMR (300 MHz, DMSO-d₆): 8.62 (1, br, 1H), 7.70-7.60 (m, 3H), 3.22 (m, 4H).

### iv. 7-(Trifluoromethyl)-2,3,4,5-tetrahydrobenzo[f][1,4]thiazepine HCl

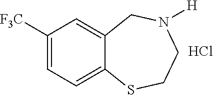 [SMILES: FC(F)(F)c1ccc2c(c1)CNCCS2 ; Cl]

¹HNMR (300 MHz, DMSO-d₆): 9.60 (br s, 1H), 8.03 (s, 1H), 7.80 (d, 1H), 7.70 (d, 1H), 4.50 (s, 2H), 3.50 (m, 2H), 3.12 (m, 2H).

### v. tert-butyl 4-(7-(trifluoromethyl)-2,3,4,5-tetrahydrobenzo[f][1,4]thiazepine-4-carbonyl)piperazine-1-carboxylate

The amine hydrochloride obtained above (1.1 mmol), DIEA (0.7 ml, 3.7 mmol) and 4-chlorocarbonyl-piperazine-1-carboxylic acid tert-butyl ester (300 mg, 1.2 mmol) were mixed in 5 ml dichloromethane. The solution was stirred at room temperature (RT) for 24 hr. The reaction solution was evaporated to dryness. The residue was dissolved in 2 ml dichloromethane and loaded onto a column. The column was washed with ethyl acetate/hexane.

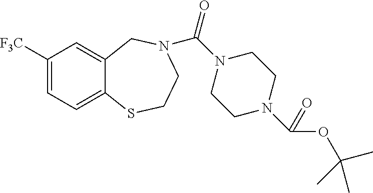 [SMILES: CC(C)(C)OC(=O)N1CCN(C(=O)N2CCSc3ccc(C(F)(F)F)cc3C2)CC1]

¹HNMR (300 MHz, CDCl3): 7.59 (m, 2H), 7.41 (d, 1H), 4.54 (s, 2H), 3.77 (m, 2H), 3.42 (m, 4H), 3.13 (m, 4H), 2.95 (m, 2H), 1.44 (s, 9H).

### vi. piperazin-1-yl(7-(trifluoromethyl)-2,3-dihydrobenzo[f][1,4]thiazepin-4(5H) yl)methanone hydrochloride (2·HCl)

To a stirred solution of tert-butyl 4-(7-(trifluoromethyl)-2,3,4,5-tetrahydrobenzo[f][1,4]thiazepine-4-carbonyl)piperazine-1-carboxylate (0.47 g, 1.05 mmol) in ether (3 ml) was added a 4 M HCl/dioxane solution (5 ml, 20 mmol). The solution was stirred at RT for 3 hrs. The solvents and excess HCl were removed, and the residue was washed with ether (10 ml) and dried on high vacuum, leaving the target product.

¹HNMR (300 MHz, DMSO-d6): 9.20 (br, 2H), 7.55 (m, 3H), 4.55 (s, 2H), 3.69 (m, 2H), 3.24 (m, 4H), 3.14 (m, 6H).

EXAMPLE 4: Compounds 3-10 were made following the general Methods 1-3 for preparing Amine (A), following by amidation according to the process of Scheme (3) or (3a).

### Ex. 4A. Piperazin-1-yl(9-(trifluoromethyl)-2,3-dihydrobenzo[f][1,4]thiazepin-4(5H)-yl)methanone hydrochloride (Compound 3·HCl)

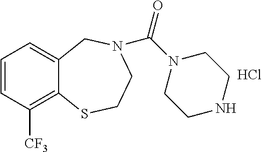 [SMILES: O=C(N1CCNCC1)N1CCSc2c(cccc2C(F)(F)F)C1 ; Cl]

Compound 3 was prepared according to the methods of Scheme (3) or (3a) path [b] and Method 1 (Scheme 4).

¹HNMR (300 MHz, DMSO-d6): 9.20 (br, 2H), 7.67 (m, 2H), 7.46 (t, 1H), 4.58 (s, 2H), 3.69 (m, 2H), 3.24 (m, 4H), 3.14 (m, 6H).

### Ex. 4B. Piperazin-1-yl(7-(trifluoromethoxy)-2,3-dihydrobenzo[f][1,4]thiazepin-4(5H)-yl)methanone hydrochloride (Compound 4·HCl)

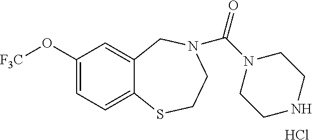 [SMILES: O=C(N1CCNCC1)N1CCSc2ccc(OC(F)(F)F)cc2C1 ; Cl]

Compound (4) was prepared according to the method of Scheme (3) or (3a) path [b] and Method 3 (Scheme 6).

1-bromo-2-(bromomethyl)-4-(trifluoromethoxy)benzene (54 mmol), trityl protected cysteamine (54 mmol) and DIEA (112 mmol) were stirred in 200 ml acetonitrile at RT for 3 hours. The solvent was removed and the residue was loaded onto column directly. The column was washed with chloroform, followed by ethyl acetate to yield the following intermediate:

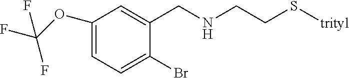

The above compound (21 g, 36.7 mmol) was dissolved in 20 ml dichloromethane and 60 ml TFA. To the solution was added triethylsilane (14 ml, 88 mmol) slowly over 30 min. The solution was stirred at RT for another 2 hour. The solvent and TFA was removed under reduced pressure. The residue was put on high vacuum pump for another 2 hours.

The above residue was mixed with CuI (700 mg, 3.7 mmol) and potassium carbonate (20 g, 145 mmol) in 150 ml iso-propanol. The mixture was stirred under reflux overnight. After cooling to RT, 300 ml dichloromethane was added and the solid was removed by filtration. The desired thiazepine was obtained by column with chloroform and methanol as eluent to form the intermediate:

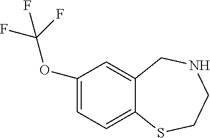 [SMILES: FC(F)(F)Oc1ccc2c(c1)CNCCS2]

¹HNMR (300 MHz, CDCl3): 7.65 (d, 1H), 7.24-7.14 (m, 2H), 4.23 (s, 2H), 3.60 (m, br, 2H), 3.05 (m, 2H).

The amine intermediate was converted to compound (4) using the general methods of Scheme (3) or (3a), path [b] as described in Example 2.

¹HNMR (300 MHz, DMSO-d₆): 9.20 (br, 2H), 7.53 (d, 1H), 7.38 (d, 1H), 7.20 (dd, 1H), 4.49 (s, 2H), 3.69 (m, 2H), 3.24 (m, 4H), 3.04 (m, 6H).

### Ex. 4C. Piperazin-1-yl(6-(trifluoromethyl)-2,3-dihydrobenzo[f][1,4]thiazepin-4(5H)-yl)methanone hydrochloride (Compound 5·HCl)

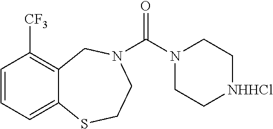

Compound (5) was prepared according to the method of Scheme (3) or (3a) path [b] and Method 2 (Scheme 5).

¹H-NMR (300 MHz, DMSO-d₆): 9.18 (br. s, 2H), 7.60 (t, 2H), 7.35 (t, 1H), 4.72 (s, 2H), 3.62 (m, 2H), 3.26 (m, 6H), 3.03 (m, 2H).

### Ex. 4D. (7,8-difluoro-2,3-dihydrobenzo[f][1,4]thiazepin-4(5H)-yl)(piperazin-1-yl)methanone hydrochloride (Compound 6·HCl)

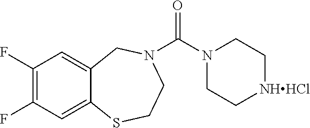

Compound (6) was prepared according to the method of Scheme (3) or (3a) path [b] and Method 3.

¹H-NMR (300 MHz, DMSO-d₆): 9.01 (br. S, 2H), 7.56-7.42+7.27-7.19 (m, 2H), 4.45 (s, 2H), 3.70-3.66 (m, 2H), 3.26-3.23 (m, 4H), 3.09 (br. S, 4H), 3.01-2.98 (m, 2H).

### Ex. 4E. (6-chloro-2,3-dihydrobenzo[f][1,4]thiazepin-4(5H)-yl)(piperazin-1-yl)methanone hydrochloride (Compound 7·HCl)

Compound (7) was prepared according to the method of Scheme (3) or (3a) path [b] and Method 2.

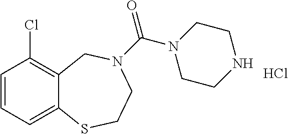 [SMILES: O=C(N1CCNCC1)N1CCSc2cccc(Cl)c2C1 ; Cl]

¹HNMR (300 MHz, DMSO-d₆): 9.17 (br, 2H), 7.32 (m, 2H), 7.10 (t, 1H), 4.69 (s, 2H), 3.64 (m, 2H), 3.28 (m, 4H), 3.17 (m, 2H), 3.04 (m, 4H).

### Ex. 4F. Piperazin-1-yl(6-(trifluoromethoxy)-2,3-dihydrobenzo[f][1,4]thiazepin-4(5H)-yl)methanone hydrochloride (Compound 8·HCl)

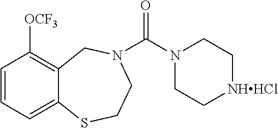

Compound (5) was prepared according to the method of Scheme (3) or (3a) path [b] and Method 3.

¹H-NMR (300 MHz, DMSO-d₆): 8.50 (br. s, 2H), 7.38 (m, 1H), 7.25 (m, 2H), 4.62 (s, 2H), 3.68 (m, 2H), 3.26 (m, 4H), 3.18 (m, 2H), 3.05 (m, 4H).

### Ex. 4G. (6-Bromo-2,3-dihydrobenzo[f][1,4]thiazepin-4(5H)-yl)(piperazin-1-yl)methanone hydrochloride (Compound 9·HCl)

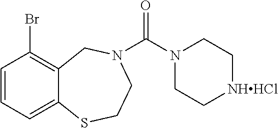

Compound (9) was prepared according to the method of Scheme (3) or (3a) path [b] and Method 2.

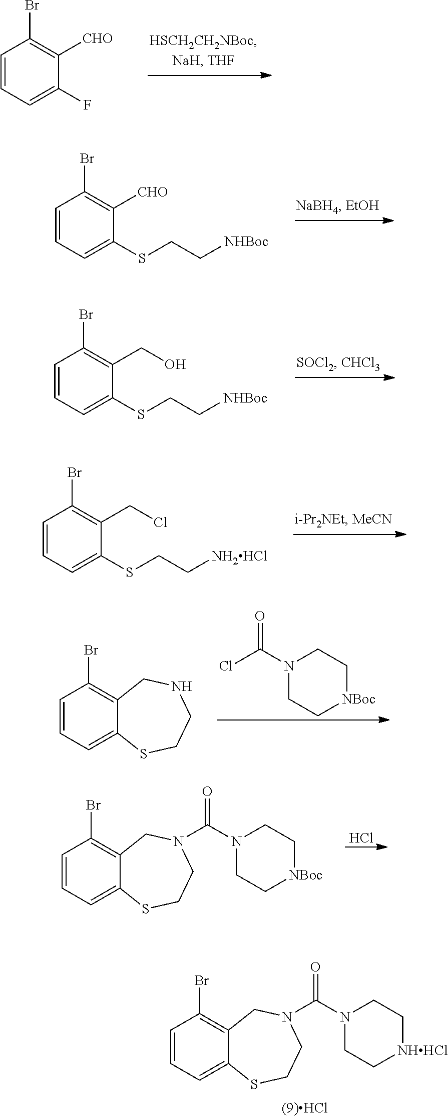

### (i) tert-Butyl 2-(3-bromo-2-formylphenylthio)ethylcarbamate

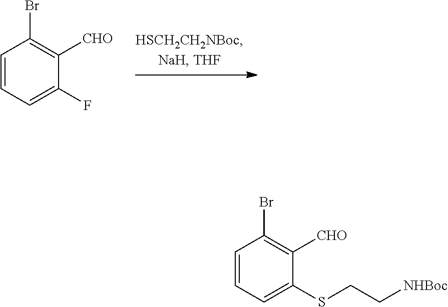 [SMILES: O=Cc1c(F)cccc1Br ; CC(C)(C)OC(=O)NCCSc1cccc(Br)c1C=O]

To a stirred suspension of NaH (60% in oil, 0.97 g, 24.14 mmol) in anhydrous THF (10 ml) at 0° C. was added dropwise a solution of 2-(Boc-amino)ethanethiol (4.09 g, 23.06 mmol) in THF (10 ml). The reaction mixture was stirred at 0° C. for 3 hrs. Then, a solution of 2-bromo-6-fluorobenzaldehyde (3.9 g, 19.21 mmol) in THF (10 ml) was added. The resulting mixture was stirred at RT overnight. After solvents were removed under reduced pressure at RT, the residue was treated with ice/water (50 ml). The product was extracted with CH₂Cl₂ (200 ml×3). The combined organic phase was dried over anhydrous sodium sulfate and filtered. Removal of solvent gave to the crude product as brown oil.

### (ii) tert-Butyl 2-(3-bromo-2-(hydroxymethyl)phenylthio)ethylcarbamate

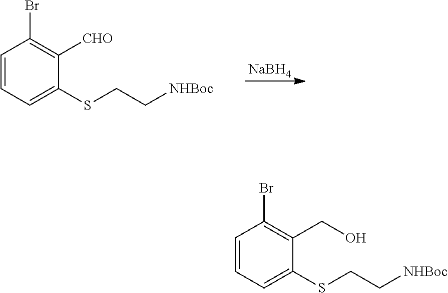 [SMILES: CC(C)(C)OC(=O)NCCSc1cccc(Br)c1C=O ; "NaBH4" ; CC(C)(C)OC(=O)NCCSc1cccc(Br)c1CO]

To a stirred solution of the crude mixture from step (i) in anhydrous ethanol (100 ml) was added NaBH₄ (778 mg, 20.57 mmol). The reaction mixture was stirred at RT for 1 hr. Then, additional NaBH₄ (2.2 g) was added and the reaction mixture was stirred at RT for another 2 hrs. After the reaction was quenched with ice/water (100 ml), the solvent ethanol was removed. The product was extracted with CH₂Cl₂ (200 ml×4). The combined organic phase was dried over anhydrous sodium sulfate and filtered. After removal of solvents, the residue was purified by column on silica gel. The desired product [tert-butyl 2-(3-bromo-2-(hydroxymethyl)phenylthio)ethylcarbamate] was obtained as a white solid.

¹H-NMR (300 MHz, CDCl₃): 7.49-7.42 (m, 2H), 7.12 (t, 1H), 5.20 (br. s, 1H,), 5.04 (d, 2H), 3.29 (q, 2H), 3.06 (t, 2H), 2.57 (t, 1H), 1.41 (s, 9H).

### (iii) 2-(3-bromo-2-(chloromethyl)phenylthio)ethanamine hydrochloride

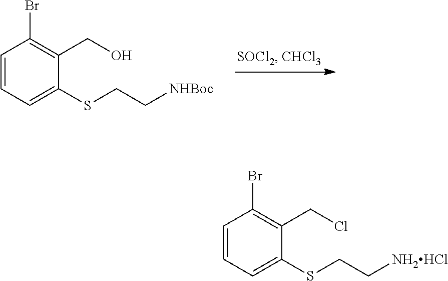

To a stirred solution of tert-butyl 2-(3-bromo-2-(hydroxymethyl) phenylthio)ethylcarbamate (3.91 g, 10.79 mmol) in CHCl₃ (30 ml) at 0° C. was added dropwise thionyl chloride (2.83 ml, 4.62 g, 38.84 mmol). The reaction mixture was stirred at 0° C. for 1 hr and at 50° C. for 5 hrs. Then, 50 ml MeOH was added at 0° C. and the mixture was stirred at RT for 1 hr. After removal of solvents, the desired crude product was obtained.

¹H-NMR (300 MHz, DMSO-d₆): 8.20 (br. s, 3H), 7.61 to 7.55 (m, 2H), 7.31 (m, 1H), 4.97 (s, 2H), 3.30 (m, 2H), 2.95 (m, 2H).

### (iv) 6-bromo-2,3,4,5-tetrahydrobenzo[f][1,4]thiazepine

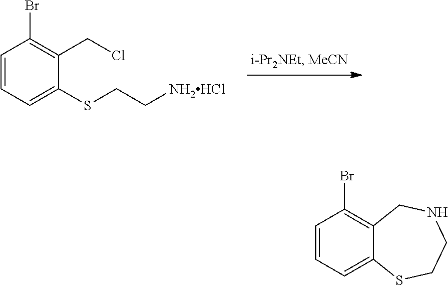

To a stirred solution of 2-(3-bromo-2-(chloromethyl)phenylthio)ethanamine hydrochloride (The crude product obtained from last step reaction, 10.79 mmol) in MeCN (300 ml) was added i-Pr₂NEt (10 ml, excess). The reaction mixture was stirred at RT overnight. After the solvents were removed, the residue was purified by column on silica gel. The desired product was obtained as a white solid.

¹H-NMR (300 MHz, CDCl₃): 7.50 (dd, 1H), 7.46 (dd, 1H), 6.96 (t, 1H), 4.43 (s, 2H), 3.38-3.34 (m, 2H), 2.85-2.81 (m, 2H).

The above product was converted to compound (9) HCl salt using the general methods described in Examples 1 and 2.

¹H-NMR (300 MHz, DMSO-d₆): 8.91 (br. s, 2H), 7.49 (dd, 1H), 7.33 (dd, 1H), 7.06 (t, 1H), 4.70 (s, 2H), 3.65-3.61 (m, 2H), 3.27-3.25 (m, 4H), 3.21-3.17 (m, 2H), 3.07 (m, 4H).

### Ex. 4H. (6-iodo-2,3-dihydrobenzo[f][1,4]thiazepin-4(5H)-yl)(piperazin-1-yl)methanone hydrochloride (Compound 10·HCl)

Compound (10) was prepared according to the method of Scheme (3) or (3a) path [b] and Method 2.

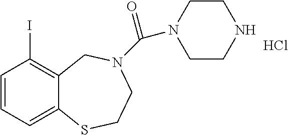 [SMILES: O=C(N1CCNCC1)N1CCSc2cccc(I)c2C1 ; Cl]

¹HNMR (300 MHz, DMSO-d₆): 9.17 (br s, 2H), 7.53 (d, 1H), 7.33 (d, 1H), 6.87 (t, 1H), 4.64 (s, 2H), 3.61 (m, 2H), 3.28 (m, 4H), 3.17 (m, 2H), 3.15 (m, 4H).

### Example 5: Inhibition of Unstimulated Calcium Release Events in Cardiomyocytes

Compounds of the disclosure were evaluated for an effect on isoproterenol-induced inhibition of unstimulated calcium release events in cardiomyocytes isolated from mouse heart.

### Experimental Protocol

Adult cardiomyocytes are isolated from female, 28-32 grams C57BL/6 mice using a perfusion solution containing Liberase TH (Sigma Aldrich, #05-401-151001). After isolation, Ca²⁺ is reintroduced in 5 steps at 4 min intervals. Then cells are plating (Time Cero) with a density of 5000-7000 rod cells in 100 μl of Cardiomyocyte Culture Medium (MEM, Invitrogen, #11575-032) containing Blebbistatin and compound treatment at various concentrations (0.01 to 10 uM). After 1 hrs of compound treatment cells are treated with Isoproterenol (1 uM). After 2 hrs of compound treatment cells are loaded with a Ca²⁺ indicator (Fluo-4-AM, Invitrogen #F14202). After 3 hrs of compound treatment MEM is quickly but gently removed and the cells re-suspended in recording solution (Ca²⁺ 2 mM, Isoproterenol 1 uM) for field stimulations using custom-designed electrodes. Cells are stimulated for 20 seconds at 0.5 Hz, then for 5 seconds at 5 Hz.

### Data Analysis:

One Study=3 experiments. One experiment=1 mouse. Each experiment has 3 controls in triplicate:
A) Ca²⁺
B) Ca²⁺+Isoproterenol
C) Ca²⁺+Isoproterenol+Control Compound.

The first step in the analysis is to determine the number of responders based on four conditions. Responders are then analyzed to determine the number of unstimulated calcium release events, which are peaks of sufficient intensity, measured in the post-5 Hz stimulation period. Effect of test compounds (Ca²⁺+Isoproterenol+Test Compound in triplicate) are expressed as a percent inhibition of the controls B vs A.

### Results:

Inhibition of unstimulated calcium release events are summarized in Table 1. All compounds were tested at 10 μM.

| Compound no. | % inhibition at 10 μM |
|---|---|
| 1 | 95-96 |
| 2 | 86-94 |
| 3 | 85 |
| 4 | 142 |
| 5 | 92 |
| 6 | 71 |
| 7 | 89 |
| 8 | 80 |
| 9 | 89 |
| 10 | 74 |

### Example 6: Bioavailability, Pharmacokinetics and Brain Exposure

Compounds of the disclosure were evaluated for oral bioavailability, pharmacokinetics and brain exposure.

### Methods

Analytical instrument: Waters Acquity Class H UPLC equipped with Waters SQ3100 mass detector.

1). Initial non-terminal assessment of compound oral bioavailability and pharmacokinetics in blood plasma.

Sprague-Dawley rats were dosed via oral gavage (20 mg/kg); blood samples were drawn at 30 min, 1 h, 4 h and 12 h and plasma concentrations were determined via UPLC/MS. The 12-hour exposure (AUC12, Table 2) was determined via trapezoidal integration of the observed concentrations.

TABLE 2
Rat plasma exposure parameters after oral administration of
compound 2. Concentration results are the average of 3 different
samples, each analyzed in duplicate via UPLC/MS.

| | Compound 2 |
|---|---|
| | PO (20 mg/kg) |
| Cₘₐₓ (ug/mL) | 0.21 |
| Cₘₐₓ (uM) | 0.61 |
| Tₘₐₓ (h) | 4 |
| AUCt (ug/mL · h) (12 h) | 2.094 |
| AUCt (uM · h) (12 h) | 6.06 |

### 2). Assessment of Brain Exposure.

Brain and plasma concentrations 4 h after oral administration (corresponding to Tmax in the first experiment) were determined via UPLC/MS, and the corresponding brain-to-plasma ratio values were calculated. Results are reported in Table 3.

TABLE 3
Plasma and brain exposure levels 4 h after oral administration compound
2 (terminal sampling). Concentration results are the average of
3 different samples, each analyzed in duplicate via UPLC/MS.

| | Compound 2 |
|---|---|
| | PO (20 mg/kg) |
| Plasma C_{4 h} (ug/mL) | 0.42 |
| Plasma C_{4 h} (uM) | 1.22 |
| Brain C_{4 h} (ug/g) | 2.16 |
| Brain C_{4 h} (uM) | 6.25 |
| Kp (brain/plasma) - 4 h | 5.1 |

3). Assessment of Compound 2 Pharmacokinetics by Intravenous (IV) Dosing

Additional PK parameters were obtained using IV dosing to complement the oral PK data and to determine absolute oral bioavailability. Compounds were administered via tail vein injection and blood samples were obtained at 30 min, 1 h, 4 h and 12h. The drug concentration in plasma was determined via UPLC/MS.

The concentration results were used to estimate PK parameters (i.e. C0, T1/2, Vd, AUCinf) based on noncompartmental distribution and mono-exponential concentration decay. The half-life value thus obtained was also used to estimate the AUCinf for the PO dosing.

A comprehensive summary of PK results following IV and po dosing, including brain-to-plasma ratio, is presented in Table 4.

TABLE 4
Rat pharmacokinetics parameters of compound 2. Concentration
results are the average of 3 different samples,
each analyzed in duplicate via UPLC/MS.

| | | Compound 2 |
|---|---|---|
| | Compound 2 | IV bolus |
| | PO (20 mg/kg) | (2 mg/kg) |
| Cmax C_{4 h} (ug/mL) | 0.32 | |
| C 1^{st} time (ug/mL) (30 min) | | 0.18 |
| Tmax (h) | 4 | |
| T½ (h) | | 2.77 |
| AUCt (ug/mL · h) (12 h) | 2.094 | |
| AUCinf (ug/mL · h) | 2.728 | 0.736 |
| Vd (L/Kg) | | 10.9 |
| F % PO/IV bolus | 37 | |
| Kp (brain/plasma) (4 hr) | 5.1 | |

### 4. Plasma and Brain Exposure

Morning and evening plasma and tissue (brain) exposure levels, measured after one week of dosing, are reported in Table 5. The exposure results after a week of dosing confirmed that compound 2 has brain exposure. Plasma and tissue exposure levels were higher in the morning than in the evening.

TABLE 5
Morning and evening rat plasma and brain exposure after
1-week administration of compound 2, each at two different
doses: 5 and 20 mg/kg/day in drinking water. Concentration
results (expressed both as μg/mL(mg) and μM) are
the Average ± Standard Deviation of 3 different samples,
each analyzed in duplicate via UPLC/MS.

| | | Brain | | Plasma | |
|---|---|---|---|---|---|
| Dose | | μg/g | μM | μg/mL | μM |
| 5 | 7:00AM | 0.50 ± 0.12 | 1.44 ± 0.36 | 0.09 ± 0.03 | 0.25 ± 0.08 |
| mg/ | 7:00PM | 0.12 ± 0.08 | 0.36 ± 0.22 | 0.02 ± 0.01 | 0.07 ± 0.02 |
| Kg | | | | | |
| 20 | 7:00AM | 3.05 ± 0.71 | 8.82 ± 2.05 | 0.42 ± 0.02 | 1.22 ± 0.05 |
| mg/ | 7:00PM | 1.20 ± 0.96 | 3.48 ± 2.79 | 0.20 ± 0.12 | 0.58 ± 0.36 |
| Kg | | | | | |

### Example 7: Binding of Calstabin2 to PKA-Phosphorylated RyR2

Binding of Calstabin2 to PKA-phosphorylated RyR2 was performed. Cardiac sarcoplasmic reticulum (CSR) was PKA-phosphorylated and incubated with Calstabin 2 at room temperature for 30 mins. The reaction was spun by centrifuge, the resulting pellet was washed, and the supernatant discarded. Proteins were separated using SDS/PAGE. Calstabin2 binding was detected with anti-Calstabin2 (primary antibody) and appropriate secondary antibody.

Compounds of the present disclosure prevented the dissociation/enhanced rebinding of Calstabin2 to PKA-phosphorylated RyR2 at a concentration of 100 nm.

### Example 8: Binding of Calstabin1 to PKA-Phosphorylated RyR1

Binding of Calstabin1 to PKA-phosphorylated RyR1 was performed in a manner similar to Example 1.

Compounds of the present disclosure prevented the dissociation/enhanced rebinding of Calstabin1 to PKA-phosphorylated RyR1 at a concentration of 100 nm.

### Example 9: Binding of Calstabin2 to RyR2 in Human Huntington Disease (HD) Cortex Microsomes

Brain microsomes were prepared from human hippocampus and cortex samples taken from HD patients. Control samples were from patients that were negative in neuropathological diagnoses.

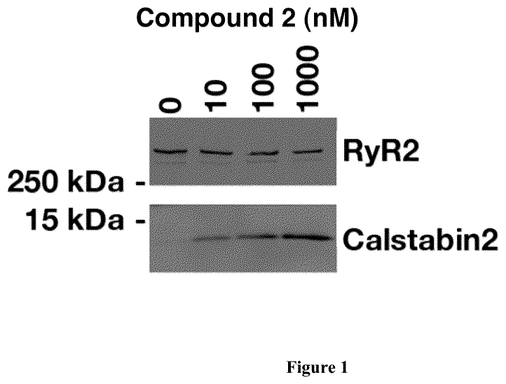

RyR2 was immunoprecipitated from Cortex lysate (+/−various concentrations of Compound 2) with an RyR2 specific antibody (2 μg) in 0.5 ml of a modified RIPA buffer (50 mM Tris-HCl pH 7.2 0.9% NaCl, 5.0 mM NaF, 1.0 mM Na₃VO₄, 1% Triton-X100, and protease inhibitors) and left overnight at 4° C. The immune complexes were incubated with protein A Sepharose beads at 4° C. for 1 h and the beads are washed three times with RIPA buffer. Immunoprecipitates were separated on SDS-PAGE gels (6% gels for RyR2, 15% gels for calstabin2) and transferred onto nitrocellulose membranes for 2h at 200 mA. Immunoblots were developed using antibodies against RyR and Calstabin. The experiment was performed using cortex lysate from a 64 year old female (36 CAG repeats) with stage 4 HD. As seen in FIG. 1, Compound 2 improves Calstabin rebinding to RyR2 (fixes channel leak) in human HD cortex microsomes).

### Example 10: Compound 2 Increases Calstabin2 to RyR2 in Human Huntington Disease (HD) Cortex Microsomes—Dose Curve

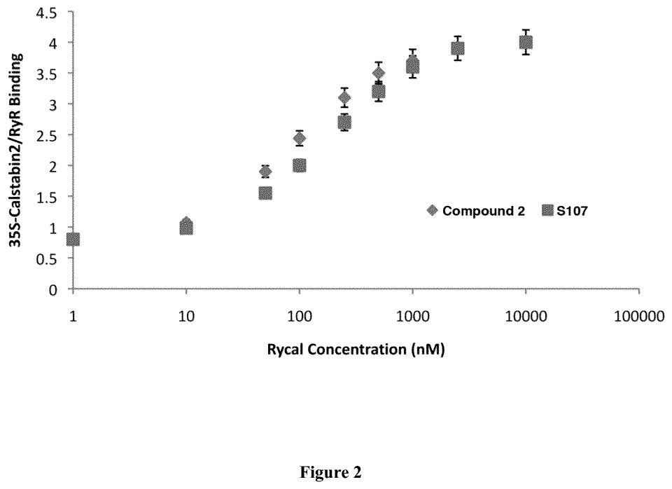

Compound 2 or Reference Rycal S107 (0-10,000 nM was added to duplicate reactions containing 150 pg of Human HD cortex microsomes. Binding reaction is initiated by addition of 10 nM ³⁵S-labelled Calstabin2. Samples are incubated at RT for 1 h. Reaction was stopped by addition of ice-cold binding buffer and then filtered through GF/B Whatman filters pre-equilibrated with 0.015% PE. Filters were washed 3 times with 5 ml of wash buffer (10 mM MOPS, 200 mM NaCl, pH 7.4), dried, and counted. Nonspecific binding was determined using 10 μM Rapamycin. As seen in FIG. 2, Compound 2 and S107 increased Calstabin2 binding to HD microsomes with an EC50=100+/−4.8 and 155+/−6.2 nM, respectively. Experiment was performed using cortex microsomes from 2 patients; a 64 year old female (36 CAG repeats) with stage 4 HD and a 63 year old male (42 CAG repeats) with stage 4 HD.

Structure of S107 is shown below.

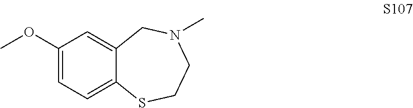

### Example 11: Compound 2 Fixes RyR Mediated Calcium Leak in Human HD Cortex Microsomes

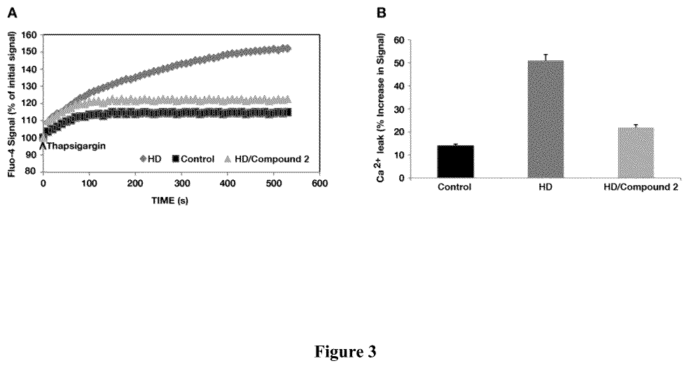

Human cortex Microsomes (5 μg/ml) were diluted into a 20 mM HEPES buffer (pH 7.2) containing 7 mM NaCl, 1.5 mM MgCl2, 120 mM K-gluconate, 5 mM K-phosphate, 8 mM K-phosphocreatine, 1 μM EGTA and 2 μM CaCl₂) mixed with 3 μM Fluo-4 and added to multiple wells of a 96-well plate. Calcium (Ca²⁺) loading of the microsomes was initiated by adding 1 mM ATP. After Ca²⁺ uptake, 3 μM thapsigargin was added. (A)—Representative traces of Ca²⁺ leak from Human cortex microsomes induced by addition of thapsigargin (3 μM). (B)—Ca²⁺ leak was calculated as the percent increase in signal after addition of thapsigargin. Data (mean±SEM) analysis was performed by Student t-test (n=2 for each group) **P<0.01. The experiment was performed using cortex microsomes from 2 patients; a 58 year old female (50 CAG repeats) with stage 4 HD and a 58 year old male (51 CAG repeats) with stage 4 HD. As seen in FIG. 3, Compound 2 decreases calcium leak from HD microsomes.

### EMBODIMENTS

Embodiment 1. A compound of Formula (I):

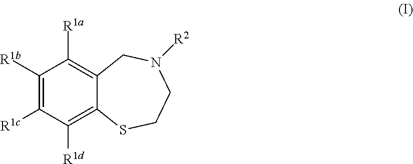

wherein
each R^{1l}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR⁷, each of which is independently substituted or unsubstituted, or hydrogen or halogen;
R² is alkyl, haloalkyl, haloalkoxy, alkenyl, alkynyl, cycloalkyl, aryl, benzyl, heteroaryl, heterocyclyl, —C(O)NR³R⁴, —C(O)C(O)NR³R⁴, —C(O)R⁸, —C(O)OR⁸, or —C(O)C(O)OR⁸, each of which is independently substituted or unsubstituted;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted; and
each R⁵, R⁶, R⁷, and R⁸ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
   or a pharmaceutically-acceptable salt thereof,
   provided that
      (a) compounds wherein (i) R^{1a}, R^{1b}, R^{1c}, and R^{1d} are each hydrogen; (ii) R^{1b} is OH or methoxy; or (iii) R² is —C(O)OtBu or —C(O)OCH₂Ph, are excluded;
      (b) when R^{1d} is methyl, then R² is not 4-methoxybenzyl; and
      (c) when R^{1a} is methyl, Cl, CN, or F, or when R^{1b} is Br, then R² is not methyl, —C(═O)H, —C(═O)Me, —C(—O)Et, or —C(═O)Ph.

Embodiment 2. The compound of embodiment 1, wherein at least one of R^{1a}, R^{1b}, R^{1c}, and R^{1d} is haloalkyl.

Embodiment 3. The compound of embodiment 1, wherein at least one of R^{1a}, R^{1b}, R^{1c}, and R^{1d} is trifluoromethyl.

Embodiment 4. The compound of embodiment 1, wherein at least one of R^{1a}, R^{1b}, R^{1c}, and R^{1d} is halogen.

Embodiment 5. The compound of embodiment 1, wherein at least one of R^{1a}, R^{1b}, R^{1c}, and R^{1d} is fluoro.

Embodiment 6. The compound of embodiment 1, wherein at least one of R^{1a}, R^{1b}, R^{1c}, and R^{1d} is chloro.

Embodiment 7. The compound of embodiment 1, wherein at least one of R^{1a}, R^{1b}, R^{1c}, and R^{1d} is bromo.

Embodiment 8. The compound of embodiment 1, wherein at least one of R^{1a}, R^{1b}, R^{1c}, and R^{1d} is iodo.

Embodiment 9. The compound of embodiment 1, wherein at least one of R^{1a}, R^{1b}, R^{1c}, and R^{1d} is haloalkoxy.

Embodiment 10. The compound of embodiment 1, wherein at least one of R^{1a}, R^{1b}, R^{1c}, and R^{1d} is trifluoromethoxy.

Embodiment 11. The compound of embodiment 1, wherein R^{1a} is trifluoromethyl.

Embodiment 12. The compound of embodiment 1, wherein R^{1b} is trifluoromethyl.

Embodiment 13. The compound of embodiment 1, wherein R^{1c} is trifluoromethyl.

Embodiment 14. The compound of embodiment 1, wherein R^{1d} is trifluoromethyl.

Embodiment 15. The compound of embodiment 1, wherein R^{1a} is trifluoromethoxy.

Embodiment 16. The compound of embodiment 1, wherein R^{1b} is trifluoromethoxy.

Embodiment 17. The compound of embodiment 1, wherein R^{1c} is trifluoromethoxy.

Embodiment 18. The compound of embodiment 1, wherein R^{1d} is trifluoromethoxy.

Embodiment 19. The compound of any one of embodiments 1-18, wherein R² is —C(O)NR³R⁴.

Embodiment 20. The compound of embodiment 19, wherein R³ and R⁴ together with the nitrogen atom to which R³ and R⁴ are attached form a heterocyclic ring, which is unsubstituted or substituted.

Embodiment 21. The compound of embodiment 19 or 20, wherein R³ and R⁴ together with the nitrogen atom to which R³ and R⁴ are attached form a piperazinyl ring, which is unsubstituted or substituted.

Embodiment 22. The compound of any one of embodiments 1-21, wherein the compound is of formula II

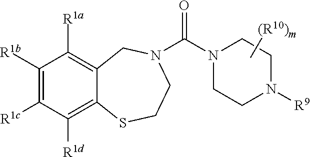

wherein
R⁹ is alkyl, alkenyl, alkynyl, cycloalkyl, aryl, benzyl, heterocyclyl, heteroaryl, —C(O)NR³R⁴, —C(O)R⁸, or —C(O)OR⁸, each of which is independently substituted or unsubstituted, or hydrogen;
each R¹⁰ is independently alkyl, alkenyl, alkynyl, cycloalkyl, aryl, benzyl, heterocyclyl, heteroaryl, —NR³R⁴, —OR⁵, or —SR⁷, each of which is unsubstituted or substituted; and
m is 0, 1, 2, 3, 4, 5, 6, 7, or 8;
   or a pharmaceutically-acceptable salt thereof.

Embodiment 23. The compound of any one of embodiments 1-22, wherein the compound is of formula III

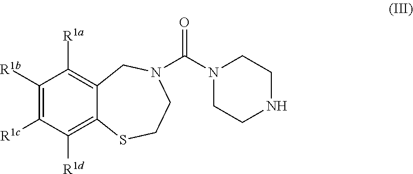

or a pharmaceutically-acceptable salt thereof.

Embodiment 24. A compound that is (6-iodo-2,3-dihydrobenzo[f][1,4]thiazepin-4(5H)-yl)(piperazin-1-yl)methanone, or a pharmaceutically-acceptable salt thereof.

Embodiment 25. A compound that is piperazin-1-yl(8-(trifluoromethyl)-2,3-dihydrobenzo [f][1,4]thiazepin-4(5H)-yl)methanone, or a pharmaceutically-acceptable salt thereof.

Embodiment 26. A compound that is piperazin-1-yl(6-(trifluoromethoxy)-2,3-dihydrobenzo [f][1,4]thiazepin-4(5H)-yl)methanone, or a pharmaceutically-acceptable salt thereof.

Embodiment 27. A compound that is (7,8-difluoro-2,3-dihydrobenzo[f][1,4]thiazepin-4(5H)-yl)(piperazin-1-yl)methanone, or a pharmaceutically-acceptable salt thereof.

Embodiment 28. The compound of any one of embodiments 1-27, wherein the pharmaceutically-acceptable salt is an acid addition salt.

Embodiment 29. The compound of any one of embodiments 1-28, wherein the pharmaceutically-acceptable salt is a hydrochloride salt.

Embodiment 30. A pharmaceutical composition comprising in a unit dosage form a compound of any one of embodiments 1-29 or a pharmaceutically-acceptable salt thereof, and a pharmaceutically-acceptable excipient.

Embodiment 31. A method of treating a condition, the method comprising administering to a subject in need thereof a therapeutically-effective amount of a compound of Formula (I):

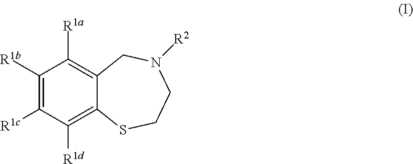

wherein
each R^{1a}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR⁷, each of which is independently substituted or unsubstituted, or hydrogen or halogen;
R² is alkyl, haloalkyl, haloalkoxy, alkenyl, alkynyl, cycloalkyl, aryl, benzyl, heteroaryl, heterocyclyl, —C(O)NR³R⁴, —C(O)C(O)NR³R⁴, —C(O)R⁸, —C(O)OR⁸, or —C(O)C(O)OR⁸, each of which is independently substituted or unsubstituted;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted; and
each R⁵, R⁶, R⁷, and R⁸ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
   or a pharmaceutically-acceptable salt thereof,
   provided that
      (a) compounds wherein (i) R^{1a}, R^{1b}, R^{1c}, and R^{1d} are each hydrogen; (ii) R^{1b} is OH or methoxy; or (iii) R² is —C(O)OtBu or —C(O)OCH₂Ph, are excluded;
      (b) when R^{1d} is methyl, then R² is not 4-methoxybenzyl; and
      (c) when R^{1a} is methyl, Cl, CN, or F, or when R^{1b} is Br, then R² is not methyl, —C(═O)H, —C(═O)Me, —C(═O)Et, or —C(═O)Ph.

Embodiment 32. The method of embodiment 31, wherein the condition is a central nervous system condition.

Embodiment 33. The method of embodiment 31 or 32, wherein the condition is essential tremor.

Embodiment 34. The method of embodiment 31 or 32, wherein the condition is a seizure.

Embodiment 35. The method of embodiment 31 or 32, wherein the condition is a neuropathy.

Embodiment 36. The method of embodiment 31 or 32, wherein the condition is post-traumatic stress disorder.

Embodiment 37. The method of embodiment 31 or 32, wherein the condition is a neurodegenerative disease.

Embodiment 38. The method of any one of embodiments 31, 32 or 37, wherein the condition is Alzheimer's disease.

Embodiment 39. The method of any one of embodiments 31, 32 or 37, wherein the condition is Huntington's disease.

Embodiment 40. The method of any one of embodiments 31, 32 or 37, wherein the condition is Amyotrophic lateral sclerosis.

Embodiment 41. The method of any one of embodiments 31, 32 or 37, wherein the condition is Spinocerebellar ataxia.

Embodiment 42. The method of any one of embodiments 31, 32 or 37, wherein the condition is Parkinson's disease.

Embodiment 43. The method of embodiment 31, wherein the condition is cognitive dysfunction.

Embodiment 44. The method of embodiment 31 or 43, wherein the condition is stress-related.

Embodiment 45. The method of embodiment 31 or 43, wherein the condition is age-related.

Embodiment 46. The method of embodiment 31 or 43, wherein the condition is memory loss.

Embodiment 47. The method of embodiment 31 or 43, wherein the condition is associated with neurodegenerative disease.

Embodiment 48. The method of embodiment 31 or 43, wherein the condition is associated with post-traumatic stress disorder.

Embodiment 49. The method of embodiment 31 or 43, wherein the condition is associated with attention deficit hyperactivity disorder.

Embodiment 50. The method of embodiment 31 or 43, wherein the condition is associated with generalized anxiety disorder.

Embodiment 51. The method of embodiment 31 or 43, wherein the condition is associated with obsessive compulsive disorder.

Embodiment 52. The method of embodiment 31 or 43, wherein the condition is associated with Schizophrenia.

Embodiment 53. The method of embodiment 31 or 43, wherein the condition is associated with Bipolar disorder.

Embodiment 54. The method of embodiment 31 or 43, wherein the condition is associated with major depression.

Embodiment 55. The method of embodiment 31, wherein the condition is a cardiac condition.

Embodiment 56. The method of embodiment 31 or 55, wherein the condition is characterized by an irregular heartbeat.

Embodiment 57. The method of any one of embodiments 31, 55 or 56, wherein the condition is catecholaminergic polymorphic ventricular tachycardia.

Embodiment 58. The method of embodiment 31 or 55, wherein the condition is heart failure.

Embodiment 59. The method of any one of embodiments 31, 55 or 58, wherein the condition is congestive heart failure.

Embodiment 60. The method of any one of embodiments 31, 55 or 58, wherein the condition is chronic heart failure.

Embodiment 61. The method of any one of embodiments 31, 55 or 58, wherein the condition is heart failure with reduced ejection fraction.

Embodiment 62. The method of any one of embodiments 31, 55 or 58, wherein the condition is heart failure with preserved ejection fraction.

Embodiment 63. The method of any one of embodiments 31, 55 or 58, wherein the subject is a heart failure patient having an implantable cardioverter-defibrillator, wherein the implantable cardioverter-defibrillator is implanted in the patient.

Embodiment 64. The method of any one of embodiments 31, 55 or 58, wherein the condition is acute heart failure.

Embodiment 65. The method of any one of embodiments 31, 55 or 58, wherein the subject is a heart failure patient in need of preservation of cardiac function post myocardial infarction.

Embodiment 66. The method of any one of embodiments 31, 55 or 58, wherein the condition is myocardial infarction.

Embodiment 67. The method of any one of embodiments 31, 55 or 58, wherein the condition comprises cardiac ischemia/reperfusion injury.

Embodiment 68. The method of embodiment 31, wherein the condition is a musculoskeletal condition.

Embodiment 69. The method of embodiment 31 or 68, wherein the condition is a congenital myopathy.

Embodiment 70. The method of any one of embodiments 31, 68 or 69, wherein the condition is RYR1-related myopathy.

Embodiment 71. The method of any one of embodiments 31 or 68-70, wherein the condition is a muscular dystrophy.

Embodiment 72. The method of any one of embodiments 31 or 68-71, wherein the condition is Duchenne Muscular Dystrophy.

Embodiment 73. The method of embodiment 31 or 68, wherein the condition is sarcopenia.

Embodiment 74. The method of embodiment 31 or 68, wherein the condition is cancer associated muscle weakness.

Embodiment 75. The method of any one of embodiments 31, 68 or 74, wherein the condition is cancer cachexia.

Embodiment 76. The method of embodiment 75, wherein the condition is cancer cachexia due to a cancer having bone metastases.

Embodiment 77. The method of embodiment 31, wherein the condition is diabetes.

Embodiment 78. The method of embodiment 31, wherein the condition is malignant hyperthermia.

Embodiment 79. The method of any one of embodiments 31-78, wherein the administering is oral.

### Additional Embodiments

Embodiment 1A. A compound represented by the structure of Formula (IV):

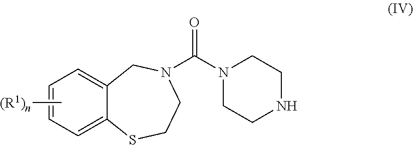

wherein
R¹ is selected from the group consisting of halogen, haloalkyl and haloalkyloxy;
n is selected from the group consisting of 1, 2, 3 and 4;
and pharmaceutically acceptable salts thereof.

Embodiment 2A. The compound according to embodiment 1A, in the form of a salt with a pharmaceutically acceptable acid or base.

Embodiment 3A. The compound according to embodiment 1A or 2A, wherein the salt is an acid addition salt.

Embodiment 4A. The compound according to any one of embodiments 1A-3A, wherein the salt is a hydrochloride salt.

Embodiment 5A. The compound according to any one of embodiments 1A-4A, wherein R¹ is haloalkyl.

Embodiment 6A. The compound according to any one of embodiments 1A-5A, wherein n is 1.

Embodiment 7A. The compound according to embodiment 1A, wherein the compound is of formula (1), or a pharmaceutically-acceptable salt thereof

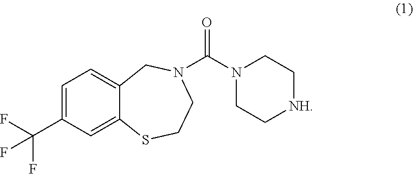 [SMILES: O=C(N1CCNCC1)N1CCSc2cc(C(F)(F)F)ccc2C1]

Embodiment 8A. The compound according to embodiment 1A, wherein the compound is of formula (2), or a pharmaceutically-acceptable salt thereof

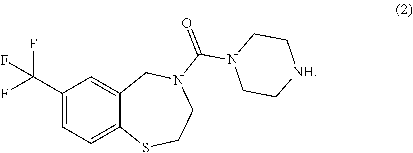 [SMILES: O=C(N1CCNCC1)N1CCSc2ccc(C(F)(F)F)cc2C1]

Embodiment 9A. The compound according to embodiment 1A, wherein the compound is of formula (3), or a pharmaceutically-acceptable salt thereof

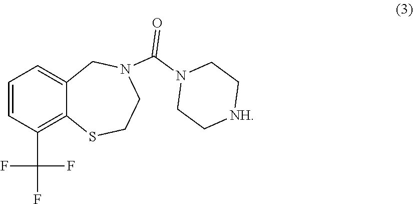 [SMILES: O=C(N1CCNCC1)N1CCSc2c(cccc2C(F)(F)F)C1]

Embodiment 10A. The compound according to embodiment 1A, wherein the compound is of formula (4), or a pharmaceutically-acceptable salt thereof

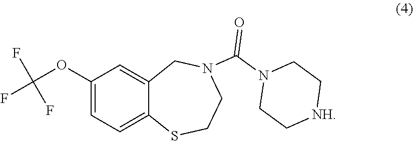 [SMILES: O=C(N1CCNCC1)N1CCSc2ccc(OC(F)(F)F)cc2C1]

Embodiment 11A. The compound according to embodiment 1A, wherein the compound is of formula (5), or a pharmaceutically-acceptable salt thereof

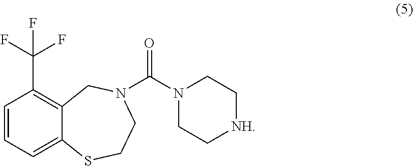 [SMILES: O=C(N1CCNCC1)N1CCSc2cccc(C(F)(F)F)c2C1]

Embodiment 12A. The compound according to embodiment 1A, wherein the compound is of formula (6), or a pharmaceutically-acceptable salt thereof

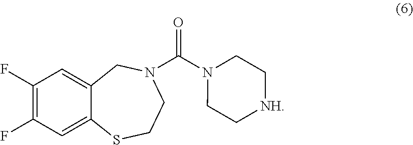 [SMILES: O=C(N1CCNCC1)N1CCSc2cc(F)c(F)cc2C1]

Embodiment 13A. The compound according to embodiment 1A, wherein the compound is of formula (7), or a pharmaceutically-acceptable salt thereof

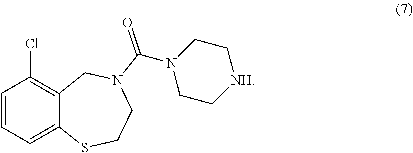 [SMILES: O=C(N1CCNCC1)N1CCSc2cccc(Cl)c2C1]

Embodiment 14A. The compound according to embodiment 1A, wherein the compound is of formula (8), or a pharmaceutically-acceptable salt thereof

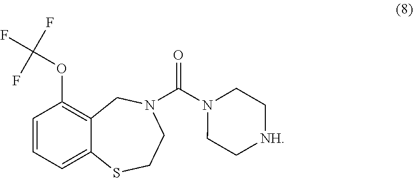 [SMILES: O=C(N1CCNCC1)N1CCSc2cccc(OC(F)(F)F)c2C1]

Embodiment 15A. The compound according to embodiment 1A, wherein the compound is of formula (9), or a pharmaceutically-acceptable salt thereof

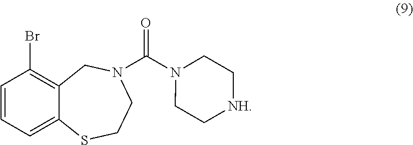 [SMILES: O=C(N1CCNCC1)N1CCSc2cccc(Br)c2C1]

Embodiment 16A. The compound according to embodiment 1A, wherein the compound is of formula (10), or a pharmaceutically-acceptable salt thereof

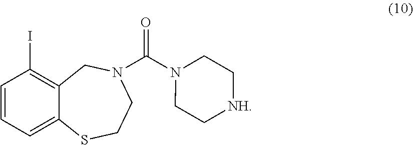 [SMILES: O=C(N1CCNCC1)N1CCSc2cccc(I)c2C1]

Embodiment 17A. A pharmaceutical composition comprising a compound according to any one of the preceding embodiments, in combination with one or more pharmaceutically acceptable excipients or carriers.

Embodiment 18A. A method of treating or preventing a condition, disease or disorder of the nervous system or, or for treating or preventing cognitive dysfunction, or for improving cognitive function, the method comprising the step of administering to a subject in need thereof a therapeutically effective amount of a compound according to any of embodiments 1A to 16A, or a pharmaceutical composition according to embodiment 17A to effectuate such treatment.

Embodiment 19A. The method according to embodiment 18A, wherein the condition, disease or disorder is associated with an abnormal function of a ryanodine receptor type 1, a ryanodine receptor type 2, a ryanodine receptor type 3, or a combination thereof.

Embodiment 20A. The method according to embodiment 18A or 19A, wherein the condition, disease or disorder is a central nervous system (CNS) or a peripheral nervous system condition, disease or disorder.

Embodiment 21A. The method according to any one of embodiments 18A-20A, wherein the condition, disease or disorder is selected from the group consisting of Alzheimer's Disease (AD), post-traumatic stress disorder (PTSD), Huntington's Disease (HD), neuropathy, seizures, Amyotrophic lateral sclerosis (ALS, Lou Gehrig's disease), Spinocerebellar ataxia (SCA), and Parkinson's Disease (PD).

Embodiment 22A. The method according to embodiment 18A, wherein the cognitive dysfunction is stress-related or age-related, or wherein the cognitive function to be improved is short term memory, long term memory, attention or learning, or wherein the cognitive dysfunction is associated with a disease or disorder selected from the group consisting of Alzheimer's disease (AD), post-traumatic stress disorder (PTSD), attention deficit hyperactivity disorder (ADHD), autism spectrum disorder (ASD), generalized anxiety disorder (GAD), obsessive compulsive disorder (OCD), Parkinson's Disease (PD), Schizophrenia, Bipolar disorder, and major depression.

Embodiment 23A. The method according to any one of embodiments 18A-22A, wherein the compound is used at a dose sufficient to restore or enhance binding of Calstabin2 to RyR2.

Embodiment 24A. The method according to any one of embodiments 18A-22A, wherein the compound is used at a dose sufficient to restore or enhance binding of Calstabin1 to RyR1.

Embodiment 25A. The method according to any one of embodiments 18A-24A, wherein the compound is used at a dose sufficient to decrease Ca²⁺ leak through a RyR channel.

Embodiment 26A. A compound according to any of embodiments 1A to 16A, or a pharmaceutically-acceptable salt thereof, or a pharmaceutical composition according to embodiment 17A, for use in the treatment or prevention of a condition, disorder or disease of the nervous system, or for treating or preventing cognitive dysfunction, or for improving cognitive function.

Embodiment 27A. The compound according to embodiment 26A, wherein the condition, disorder or disease is a central nervous system (CNS) or a peripheral nervous system condition, disorder or disease.

Embodiment 28A. A process for the preparation of a compound according to any of claims 1A to 16A, comprising the steps of
(a) reacting a compound of formula A′

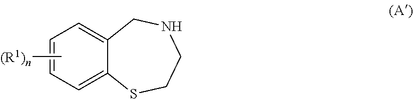

with an acylating agent to generate a compound of formula (B′):

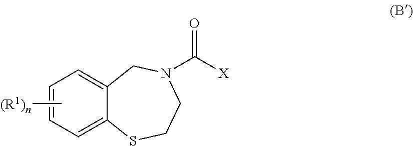

wherein R¹ and n are as defined in claim 1 and X is a leaving group;
(b) reacting compound (B′) with an optionally protected piperazine to generate a compound of formula (C″)

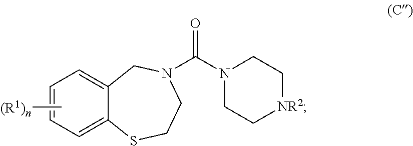

wherein R² is H or a nitrogen protecting group; and
(c) optionally, removing the nitrogen protecting group to generate a compound of formula (IV).

Embodiment 29A. A process for the preparation of a compound according to any of claims 1A to 16A, comprising the steps of
(a) reacting a compound of formula A′

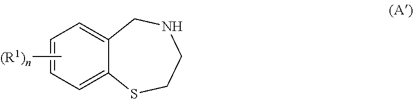

with an acylated piperazine derivative of the formula

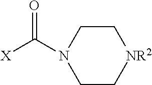

to generate a compound of formula (C″)

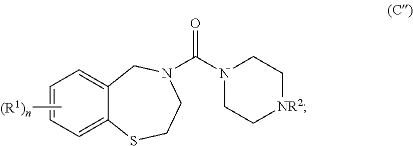

wherein R² is H or a nitrogen protecting group; and
(b) optionally, removing the nitrogen protecting group to generate a compound of formula (IV).

Embodiment 30A. A process for the preparation of a compound of formula (I′)

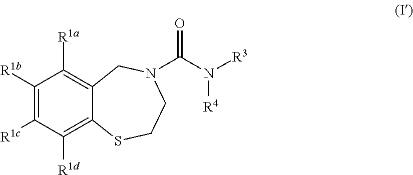

wherein
each R^{1a}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —ORV, —SO₃H, —SO₂R⁶, —OSO₂R⁶, —S(O)R⁶, or —SR⁷, each of which is independently substituted or unsubstituted, or hydrogen or halogen;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted; and
each R⁵, R⁶, and R⁷ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
or a pharmaceutically-acceptable salt thereof,
   the process comprising the steps of:
      (a) reacting a compound of formula A

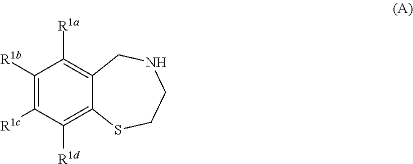

with an acylating agent to generate a compound of formula (B):

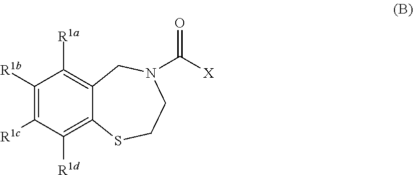

wherein X is a leaving group;
(b) reacting compound (B) with an amine of formula NHR^{3a}R^{4a} to generate a compound of formula (C):

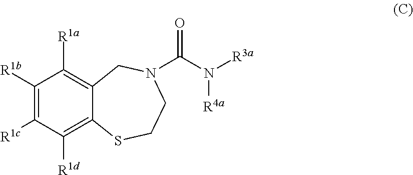

wherein R^{3a} is R³ or a nitrogen protecting group; and R^{4a} is R⁴ or a nitrogen protecting group; and
   (c) optionally, removing the nitrogen protecting group to generate a compound of formula (I′).

Embodiment 31A. A process for the preparation of a compound of formula (I′)

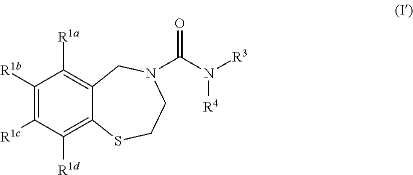

wherein
each R^{1a}, R^{1b}, R^{1c}, and R^{id} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR⁷, each of which is independently substituted or unsubstituted, or hydrogen or halogen;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted; and
each R⁵, R⁶, and R⁷ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
   or a pharmaceutically-acceptable salt thereof,
the process comprising the steps of:
   (a) reacting a compound of formula A

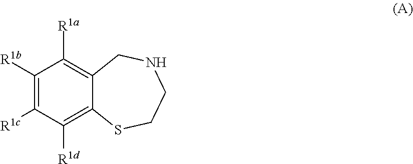

with a compound of formula

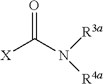

wherein X is a leaving group, R^{3a} is R³ or a nitrogen protecting group, and R^{4a} is R⁴ or a nitrogen protecting group; to generate a compound of formula (C):

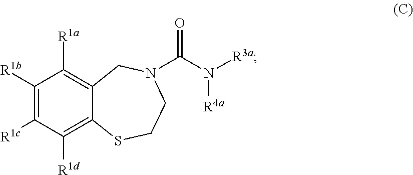

and
   (b) optionally, removing the nitrogen protecting group to generate a compound of formula

Embodiment 32A. A process for the preparation of a compound of formula (II)

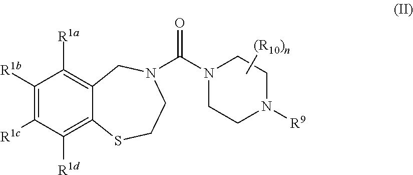

wherein
each R^{1a}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR⁷, each of which is independently substituted or unsubstituted, or hydrogen or halogen;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted;
each R⁵, R⁶, and R⁷ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
R⁹ is alkyl, alkenyl, alkynyl, cycloalkyl, aryl, benzyl, heterocyclyl, heteroaryl, —C(O)NR³R⁴, —C(O)R⁸, or —C(O)OR⁸, each of which is independently substituted or unsubstituted, or hydrogen;
each R¹⁰ is independently alkyl, alkenyl, alkynyl, cycloalkyl, aryl, benzyl, heterocyclyl, heteroaryl, —NR³R⁴, —OR⁵, or —SR⁷, each of which is unsubstituted or substituted; and
m is 0, 1, 2, 3, 4, 5, 6, 7, or 8;
   or a pharmaceutically-acceptable salt thereof,
   comprising the steps of
(a) reacting a compound of formula A

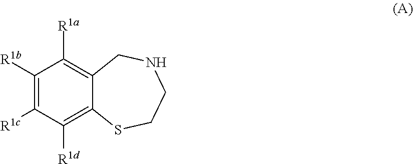

with an acylating agent to generate a compound of formula (B):

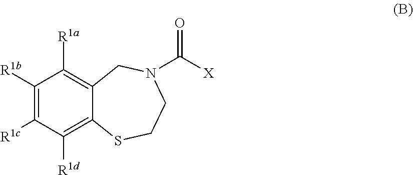

wherein X is a leaving group;
(b) reacting compound (B) with an amine of formula

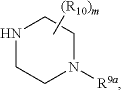

wherein R^{9a} is R⁹ or a nitrogen leaving group, to generate a compound of formula (C′):

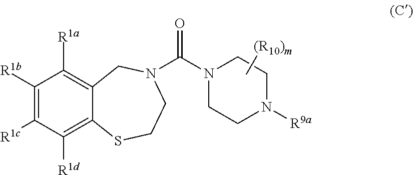

and
(c) optionally, removing the nitrogen protecting group to generate a compound of formula (II).

Embodiment 33A. A process for the preparation of a compound of formula (II)

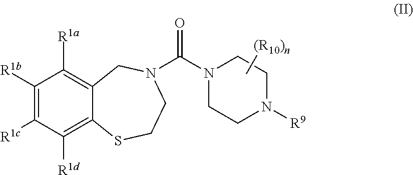

wherein
each R^{1a}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR⁷, each of which is independently substituted or unsubstituted, or hydrogen or halogen;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted;
each R⁵, R⁶, and R⁷ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
R⁹ is alkyl, alkenyl, alkynyl, cycloalkyl, aryl, benzyl, heterocyclyl, heteroaryl, —C(O)NR³R⁴, —C(O)R⁸, or —C(O)OR⁸, each of which is independently substituted or unsubstituted, or hydrogen;
each R¹⁰ is independently alkyl, alkenyl, alkynyl, cycloalkyl, aryl, benzyl, heterocyclyl, heteroaryl, —NR³R⁴, —OR⁵, or —SR⁷, each of which is unsubstituted or substituted; and
m is 0, 1, 2, 3, 4, 5, 6, 7, or 8;
   or a pharmaceutically-acceptable salt thereof,
   comprising the steps of
(a) reacting a compound of formula A

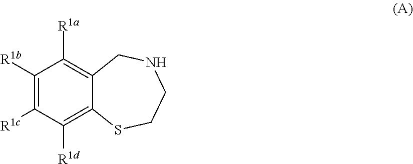

with a compound of formula

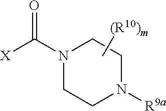

wherein X is a leaving group and R^{9a} is R⁹ or a nitrogen leaving group, to generate a compound of formula (C′):

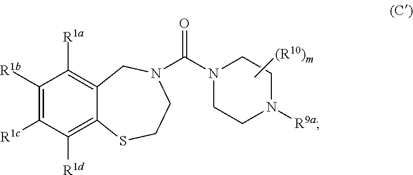

and
(b) optionally, removing the nitrogen protecting group to generate a compound of formula (II).

Embodiment 34A. A process for the preparation of a compound of formula (III)

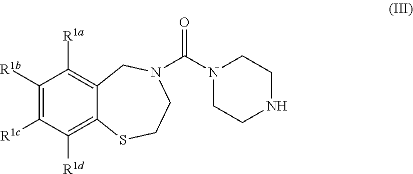

wherein
each R^{1a}, R^{1b}, R^{1c}, and R^{id} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR⁷, each of which is independently substituted or unsubstituted, or hydrogen or halogen;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted;
each R⁵, R⁶, and R⁷ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
   or a pharmaceutically-acceptable salt thereof,
   comprising the steps of
(a) reacting a compound of formula A

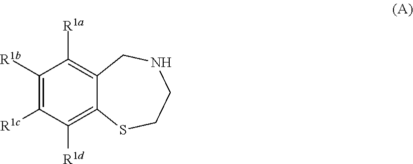

with a compound of formula

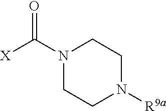

wherein X is a leaving group, and R^{9a} is hydrogen or a nitrogen protecting group; to generate a compound of formula (C′);

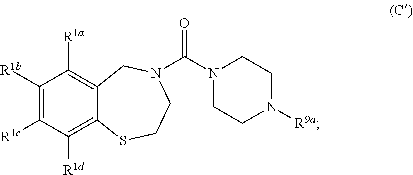

and
(b) when R^{9a} is a nitrogen protecting group, removing the nitrogen protecting group to generate a compound of formula (III).

Embodiment 35A. A process for the preparation of a compound of formula (III)

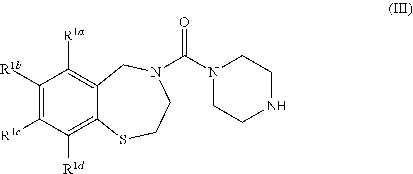

wherein
each R^{1a}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR′, each of which is independently substituted or unsubstituted, or hydrogen or halogen;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted;
each R⁵, R⁶, and R⁷ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
   or a pharmaceutically-acceptable salt thereof;
   comprising the steps of
(a) reacting a compound of formula A

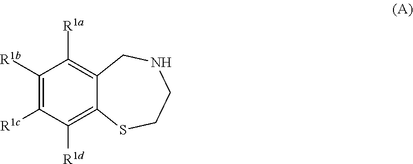

with an acylating agent to generate a compound of formula (B):

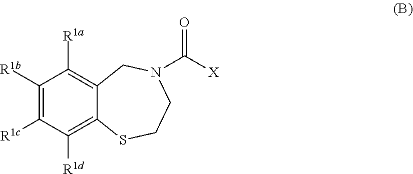

wherein X is a leaving group; and
(b) reacting compound (B) with an optionally protected piperazine of formula

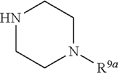

wherein X is a leaving group, and R^{9a} is hydrogen or a nitrogen protecting group, to generate a compound of formula (C′);

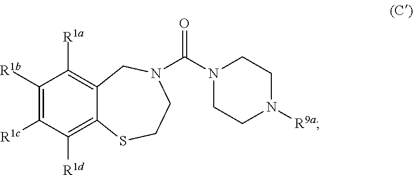

(c) when R^{9a} is a nitrogen protecting group, removing the nitrogen protecting group to generate a compound of formula (III).

1. A compound of Formula (I): 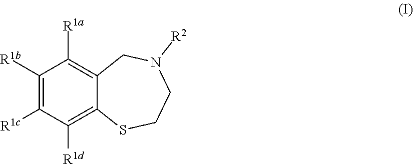
wherein
each R^{1a}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR⁷, each of which is independently substituted or unsubstituted, or hydrogen or halogen;
R² is alkyl, haloalkyl, haloalkoxy, alkenyl, alkynyl, cycloalkyl, aryl, benzyl, heteroaryl, heterocyclyl, —C(O)NR³R⁴, —C(O)C(O)NR³R⁴, —C(O)R⁸, —C(O)OR⁸, or —C(O)C(O)OR⁸, each of which is independently substituted or unsubstituted;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted; and
each R⁵, R⁶, R⁷, and R⁸ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
or a pharmaceutically-acceptable salt thereof,
provided that
(a) compounds wherein (i) R^{1a}, R^{1b}, R^{1c}, and R^{1d} are each hydrogen; (ii) R^{1b} is OH or methoxy; or (iii) R² is —C(O)OtBu or —C(O)OCH₂Ph, are excluded;
(b) when R^{1d} is methyl, then R² is not 4-methoxybenzyl; and
(c) when R^{1a} is methyl, Cl, CN, or F, or when R^{1b} is Br, then R² is not methyl, —C(═O)H, —C(═O)Me, —C(═O)Et, or —C(═O)Ph.

2. The compound of claim 1, wherein at least one of R^{1a}, R^{1b}, R^{1c}, and R^{1d} is haloalkyl.

3. The compound of claim 1, wherein at least one of R^{1a}, R^{1b}, R^{1c}, and R^{1d} is trifluoromethyl.

4. The compound of claim 1, wherein at least one of R^{1a}, R^{1b}, R^{1c}, and R^{1d} is halogen.

5-8. (canceled)

9. The compound of claim 1, wherein at least one of R^{1a}, R^{1b}, R^{1c}, and R^{1d} is haloalkoxy.

10. The compound of claim 1, wherein at least one of R^{1a}, R^{1b}, R^{1c}, and R^{1d} is trifluoromethoxy.

11. The compound of claim 1, wherein R^{1a} is trifluoromethyl.

12. The compound of claim 1, wherein R^{1b} is trifluoromethyl.

13. The compound of claim 1, wherein R^{1c} is trifluoromethyl.

14. The compound of claim 1, wherein R^{1d} is trifluoromethyl.

15-18. (canceled)

19. The compound of claim 1, wherein R² is —C(O)NR³R⁴.

20. The compound of claim 0, wherein R³ and R⁴ together with the nitrogen atom to which R³ and R⁴ are attached form a heterocyclic ring, which is unsubstituted or substituted.

21. (canceled)

22. The compound of claim 1, wherein the compound is of formula II 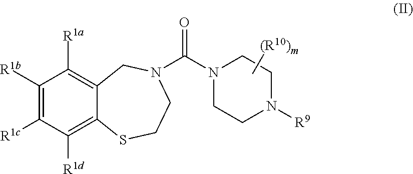
wherein
R⁹ is alkyl, alkenyl, alkynyl, cycloalkyl, aryl, benzyl, heterocyclyl, heteroaryl, —C(O)NR³R⁴, —C(O)R⁸, or —C(O)OR⁸, each of which is independently substituted or unsubstituted, or hydrogen;
each R¹⁰ is independently alkyl, alkenyl, alkynyl, cycloalkyl, aryl, benzyl, heterocyclyl, heteroaryl, —NR³R⁴, —OR⁵, or —SR⁷, each of which is unsubstituted or substituted; and
m is 0, 1, 2, 3, 4, 5, 6, 7, or 8;
or a pharmaceutically-acceptable salt thereof.

23. The compound of claim 1, wherein the compound is of formula III 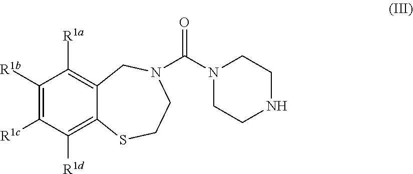
or a pharmaceutically-acceptable salt thereof.

24. (canceled)

25. The compound of claim 1, wherein the compound is piperazin-1-yl(8-(trifluoromethyl)-2,3-dihydrobenzo[f][1,4]thiazepin-4(5H)-yl)methanone, or a pharmaceutically-acceptable salt thereof.

26. The compound of claim 1, wherein the compound is piperazin-1-yl(6-(trifluoromethoxy)-2,3-dihydrobenzo[f][1,4]thiazepin-4(5H)-yl)methanone, or a pharmaceutically-acceptable salt thereof.

27. The compound of claim 1, wherein the compound is (7,8-difluoro-2,3-dihydrobenzo[f][1,4]thiazepin-4(5H)-yl)(piperazin-1-yl)methanone, or a pharmaceutically-acceptable salt thereof.

28. The compound of claim 1, wherein the compound is piperazin-1-yl(7-(trifluoromethyl)-2,3-dihydrobenzo[f][1,4]thiazepin-4(5H)-yl)methanone, or a pharmaceutically-acceptable salt thereof.

29. (canceled)

30. The compound of claim 1, wherein the compound is piperazin-1-yl(7-(trifluoromethoxy)-2,3-dihydrobenzo[f][1,4]thiazepin-4(5H)-yl)methanone, or a pharmaceutically-acceptable salt thereof.

31-36. (canceled)

37. A method of treating a condition, the method comprising administering to a subject in need thereof a therapeutically-effective amount of a compound of Formula (I): 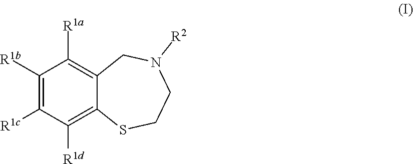
wherein
each R^{1a}, R^{1b}, R^{1c}, and R^{1d} is independently alkyl, haloalkyl, haloalkoxy, alkenyl, alkynyl, cycloalkyl, aryl, benzyl, heteroaryl, heterocyclyl, —CN, —NO₂, —N₃, —NR³R⁴, —OR⁵, —SO₃H, —SO₂R⁶, —OSO₂R⁶, —S(O)R⁶, or —SR⁷, each of which is independently substituted or unsubstituted, or hydrogen or halogen;
R² is alkyl, haloalkyl, haloalkoxy, alkenyl, alkynyl, cycloalkyl, aryl, benzyl, heteroaryl, heterocyclyl, —C(O)NR³R⁴, —C(O)C(O)NR³R⁴, —C(O)R⁸, —C(O)OR⁸, or —C(O)C(O)OR⁸, each of which is independently substituted or unsubstituted;
each R³ and R⁴ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen; or R³ and R⁴ together with the nitrogen atom to which R³ and R⁴ are attached form a heterocyclic or heteroaromatic ring, which is unsubstituted or substituted; and
each R⁵, R⁶, R⁷, and R⁸ is independently alkyl, haloalkyl, haloalkoxy, alkenyl, alkynyl, cycloalkyl, aryl, benzyl, heteroaryl, or heterocyclyl, each of which is independently substituted or unsubstituted, or hydrogen or halogen;
or a pharmaceutically-acceptable salt thereof,
provided that
(a) compounds wherein (i) R^{1a}, R^{1b}, R^{1c}, and R^{1d} are each hydrogen; (ii) R^{1b} is OH or methoxy; or (iii) R² is —C(O)OtBu or —C(O)OCH₂Ph, are excluded;
(b) when R^{1d} is methyl, then R² is not 4-methoxybenzyl, and
(c) when R^{1a} is methyl, Cl, CN, or F, or when R^{b} is Br, then R² is not methyl, —C(═O)H, —C(═O)Me, —C(═O)Et, or —C(═O)Ph.

38. The method of claim 37, wherein the condition is a central nervous system condition.

39-60. (canceled)

61. The method of claim 37, wherein the condition is a cardiac condition.

62. (canceled)

63. The method of claim 37, wherein the condition is catecholaminergic polymorphic ventricular tachycardia.

64. The method of claim 37, wherein the condition is heart failure.

65-75. (canceled)

76. The method of claim 37, wherein the condition is RYR1-related myopathy.

77-85. (canceled)
